(19) United States
(12) Patent Application Publication
(10) Pub. No.: US 2026/0224157 A1
(43) Pub. Date: Aug. 6, 2026

(54) SIGNAL PROCESSING SYSTEM

(71) Applicant: SONY GROUP CORPORATION, TOKYO (JP)

(72) Inventors: RIHO HITSUYU, TOKYO (JP); YASUHIDE HYODO, TOKYO (JP); MAO KATSUHARA, TOKYO (JP)

(21) Appl. No.: 19/128,739

(22) PCT Filed: Nov. 9, 2023
(86) PCT No.: PCT/JP2023/040368

§ 371 Date: May 9, 2025

(30) Foreign Application Priority Data
Nov. 16, 2022 (JP) 2022-183356

(51) Int. Cl. A61B 5/374; A61B 5/16

(52) U.S. Cl. CPC A61B 5/374; A61B 5/165

(57) ABSTRACT

A signal processing system according to the present disclosure includes: a signal analysis unit that calculates a blinking component indicator and a brain wave signal feature amount on a basis of a blinking component and a brain wave component included in a brain wave signal actually measured by an electroencephalograph; a correction unit that performs a correction of the blinking component indicator or the brain wave signal feature amount in the actually measured brain wave signal on a basis of a correction table generated on a basis of a brain wave signal serving as a reference for the correction, the blinking component being superimposed on the brain wave signal feature amount; and an emotion estimation unit that performs an emotion estimation on a basis of at least the corrected blinking component indicator or the corrected brain wave signal feature amount on which the blinking component is superimposed.

### TECHNICAL FIELD

The present disclosure relates to a signal processing system.

### BACKGROUND ART

There is a technique of detecting, as noise, a blinking component and the like superimposed on a brain wave signal acquired by an electroencephalograph (Electroencephalograph (EEG)) (see PTL 1). There is further a technique of estimating a wakefulness level as an emotion (see PTL 2).

### CITATION LIST

### Patent Literature

PTL 1: Japanese Unexamined Patent Application Publication No. 2008-168148

PTL 2: Japanese Unexamined Patent Application Publication No. 2009-261516

### SUMMARY OF THE INVENTION

It is conceivable that a blinking component superimposed on a brain wave signal acquired by an electroencephalograph is not regarded as noise but is utilized for an emotion estimation. In this case, a blinking component indicator such as an intensity or a waveform of the blinking component changes depending on a difference in measurement environment. Further, in experiencing content such as a game, a relation between blinking and an emotion changes depending on a difference in context, which decreases accuracy of the emotion estimation.

Accordingly, it is desirable to provide a signal processing system that makes it possible to utilize, regardless of a difference in measurement environment, a blinking component superimposed on a brain wave signal for an emotion estimation. Further, it is desirable to provide a signal processing system that makes it possible to perform the emotion estimation in consideration of an influence of a context.

A first signal processing system according to an embodiment of the present disclosure includes: a signal analysis unit that calculates a blinking component indicator and a brain wave signal feature amount on a basis of a blinking component and a brain wave component included in a brain wave signal actually measured by an electroencephalograph; a correction unit that performs a correction of the blinking component indicator or the brain wave signal feature amount in the actually measured brain wave signal on a basis of a correction table generated on a basis of a brain wave signal serving as a reference for the correction, the blinking component being superimposed on the brain wave signal feature amount; and an emotion estimation unit that performs an emotion estimation on a basis of at least the corrected blinking component indicator or the corrected brain wave signal feature amount on which the blinking component is superimposed.

A second signal processing system according to an embodiment of the present disclosure includes a second information processing apparatus that performs an emotion estimation on a basis of a signal outputted from a first information processing apparatus, the first information processing apparatus including a signal analysis unit that calculates a blinking component indicator and a brain wave signal feature amount on a basis of a blinking component and a brain wave component included in a brain wave signal actually measured by an electroencephalograph, and a correction unit that performs a correction of the blinking component indicator or the brain wave signal feature amount in the actually measured brain wave signal on a basis of a correction table generated on a basis of a brain wave signal serving as a reference for the correction, the blinking component being superimposed on the brain wave signal feature amount, and the second information processing apparatus including an emotion estimation unit that performs the emotion estimation on a basis of at least the corrected blinking component indicator or the corrected brain wave signal feature amount.

A third signal processing system according to an embodiment of the present disclosure includes a second information processing apparatus that performs an emotion estimation on a basis of a signal outputted from a first information processing apparatus, the first information processing apparatus including a signal analysis unit that calculates a blinking component indicator and a brain wave signal feature amount on a basis of a blinking component and a brain wave component included in a brain wave signal actually measured by an electroencephalograph, and the second information processing apparatus including a correction unit that performs a correction of the blinking component indicator or the brain wave signal feature amount in the actually measured brain wave signal on a basis of a correction table generated on a basis of a brain wave signal serving as a reference for the correction, the blinking component being superimposed on the brain wave signal feature amount, and an emotion estimation unit that performs the emotion estimation on a basis of at least the corrected blinking component indicator or the corrected brain wave signal feature amount.

In any of the first to third signal processing systems according to the embodiments of the present disclosure, the blinking component indicator and the brain wave signal feature amount are calculated on the basis of the blinking component and the brain wave component included in the brain wave signal actually measured by the electroencephalograph. Further, the correction of the blinking component indicator or the brain wave signal feature amount on which the blinking component is superimposed in the actually measured brain wave signal is performed on the basis of the correction table generated on the basis of the brain wave signal serving as the reference for the correction. The emotion estimation is performed on the basis of at least the corrected blinking component indicator or the corrected brain wave signal feature amount on which the blinking component is superimposed.

Further, a fourth signal processing system according to an embodiment of the present disclosure includes a selection unit that selects a correction method of an emotion estimation on a basis of information regarding content, the correction method being in accordance with a context; and an emotion estimation unit that performs the emotion estimation based on a blinking component indicator calculated on a basis of a blinking signal or a brain wave signal, in which the emotion estimation unit performs the emotion estimation based on the blinking component indicator through the correction method selected by the selection unit.

In the fourth signal processing system according to the embodiment of the present disclosure, the correction method of the emotion estimation in accordance with the context is selected on the basis of the information regarding the content, and the emotion estimation based on the blinking component indicator is performed through the selected correction method.

### BRIEF DESCRIPTION OF DRAWINGS

FIG. 1 is a block diagram schematically illustrating a configuration example of a signal processing system according to a first embodiment of the present disclosure.

FIG. 2 is an explanatory graph illustrating an example of content of a correction table to be used for a correction to be performed by a blinking correction unit in the signal processing system according to the first embodiment.

FIG. 3 is an explanatory diagram illustrating a first example of a correction method to be performed by the blinking correction unit in the signal processing system according to the first embodiment.

FIG. 4 is an explanatory diagram illustrating a second example of the correction method to be performed by the blinking correction unit in the signal processing system according to the first embodiment.

FIG. 5 is a block diagram schematically illustrating a configuration example of a signal processing system according to Modification Example 1 of the first embodiment.

FIG. 6 is an explanatory graph illustrating an example of a correction method to be performed by a blinking correction unit in the signal processing system according to Modification Example 1 of the first embodiment.

FIG. 7 is a block diagram schematically illustrating a configuration example of a signal processing system according to Modification Example 2 of the first embodiment.

FIG. 8 is a configuration example schematically illustrating a configuration example of a signal processing system according to Modification Example 3 of the first embodiment.

FIG. 9 is a block diagram schematically illustrating a configuration example of a signal processing system according to Modification Example 4 of the first embodiment.

FIG. 10 is an explanatory graph illustrating an example of a method of separating a blinking component from a brain wave signal feature amount with a feature amount correction section in the signal processing system according to Modification Example 4 of the first embodiment.

FIG. 11 is a block diagram schematically illustrating a first configuration example of a signal processing system according to Modification Example 5 of the first embodiment.

FIG. 12 is a block diagram schematically illustrating a second configuration example of the signal processing system according to Modification Example 5 of the first embodiment.

FIG. 13 is a block diagram schematically illustrating a third configuration example of the signal processing system according to Modification Example 5 of the first embodiment.

FIG. 14 is a block diagram schematically illustrating a configuration example (Specific Example 1) of a signal processing system according to a second embodiment.

FIG. 15 is an explanatory graph schematically illustrating an example of a relationship between blinking and a wakefulness level.

FIG. 16 is an explanatory diagram schematically illustrating an example of the relationship between the blinking and the wakefulness level in each context.

FIG. 17 is a flowchart illustrating an example of content of processing to be performed by the signal processing system according to Specific Example 1 using genre information as a trigger.

FIG. 18 is a flowchart illustrating an example of content of processing to be performed by the signal processing system according to Specific Example 1 using scene information as a trigger.

FIG. 19 is a flowchart illustrating an example of content of processing to be performed by the signal processing system according to Specific Example 1 using scene switching as a trigger.

FIG. 20 is a flowchart illustrating an example of content of processing to be performed by the signal processing system according to Specific Example 1 using playtime as a trigger.

FIG. 21 is a block diagram schematically illustrating a configuration example (Specific Example 2) of the signal processing system according to the second embodiment.

FIG. 22 is a block diagram schematically illustrating a configuration example (Specific Example 3) of the signal processing system according to the second embodiment.

FIG. 23 is a block diagram schematically illustrating a configuration example (Specific Example 4) of the signal processing system according to the second embodiment.

FIG. 24 is a block diagram schematically illustrating a configuration example (Specific Example 5) of the signal processing system according to the second embodiment.

FIG. 25 is a block diagram schematically illustrating a configuration example (Specific Example 6) of the signal processing system according to the second embodiment.

FIG. 26 is a block diagram schematically illustrating a configuration example (a first configuration example of Specific Example 7) of the signal processing system according to the second embodiment.

FIG. 27 is a block diagram schematically illustrating a configuration example (a second configuration example of Specific Example 7) of the signal processing system according to the second embodiment.

FIG. 28 is a block diagram schematically illustrating a configuration example (a third configuration example of Specific Example 7) of the signal processing system according to the second embodiment.

FIG. 29 is a block diagram schematically illustrating a configuration example (a fourth configuration example of Specific Example 7) of the signal processing system according to the second embodiment.

### MODES FOR CARRYING OUT THE INVENTION

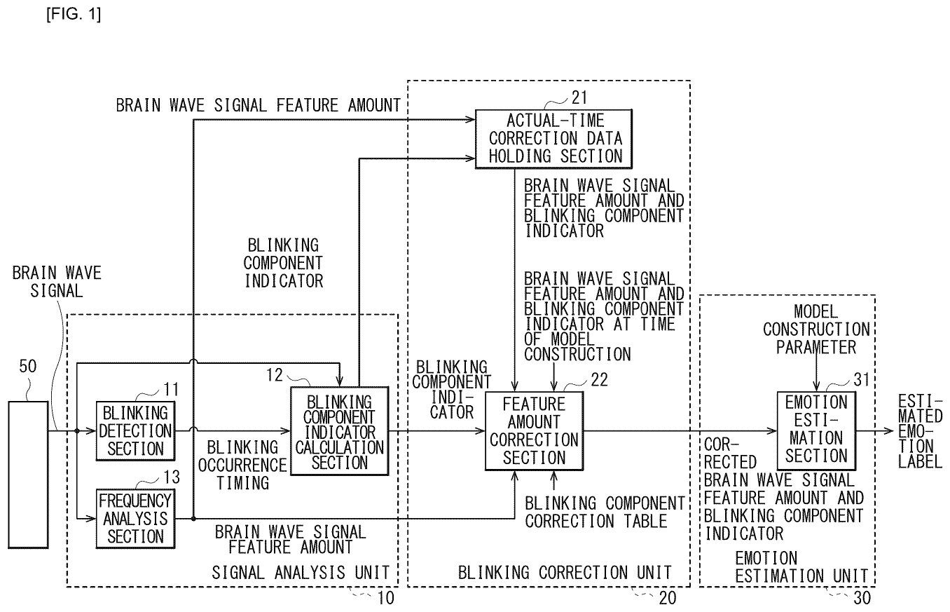

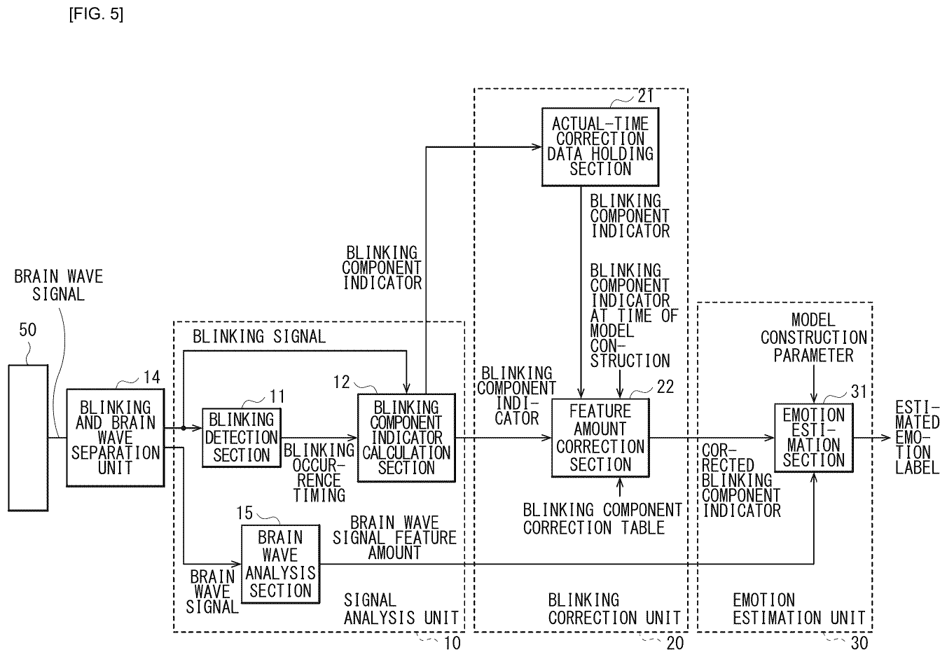

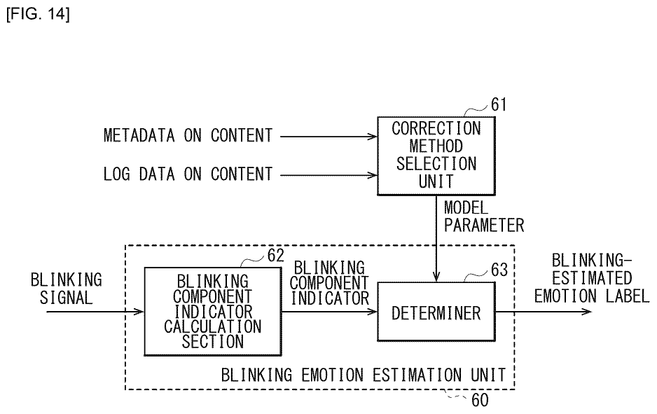

Hereinafter, description is given in detail of embodiments of the present disclosure with reference to the drawings. It is to be noted that the description is given in the following order.
0. Comparative Example
1. First Embodiment (FIGS. 1 to 4)
   1.1 Configuration
   1.2 Operation
   1.3 Effects
2. Modification Examples of First Embodiment (FIGS. 5 to 13)
   2.1 Modification Example 1
   2.2 Modification Example 2
   2.3 Modification Example 3
   2.4 Modification Example 4
   2.5 Modification Example 5
3. Second Embodiment (FIGS. 14 to 29)
   3.1 Overview
   3.2 Specific Examples
   3.3 Effects
4. Other Embodiments

### 0. COMPARATIVE EXAMPLE

Various biological modalities (various types of biological information) related to a wakefulness level can be acquired from a human frontal region. In particular, a frontal brain wave is known to be related to the wakefulness level. Further, a blinking component is known to contribute to an attention and wakefulness state, and may be context information utilizable in addition to a brain wave in accordance with an application. In view of these facts, presumably an improvement in accuracy of an emotion estimation is expectable if the blinking component is utilizable as a context in the emotion estimation with the brain wave. As a common technique, some already existing cases achieve an improvement in accuracy of emotion discrimination using a brain wave component and a blinking component. In these cases, however, it is typical to use a device or an instrument other than an electroencephalograph, such as an eye tracker or an electrooculograph. When a user acquires various biological modalities and utilizes the various biological modalities for an estimation of the wakefulness level in a daily living environment, necessity of a device or an instrument other than the electroencephalograph leads to narrowing a scope of use of the application.

Meanwhile, in measuring the frontal brain wave with the electroencephalograph, not only the brain wave component but also the blinking component is superimposed on the acquired brain wave signal. In a common technique, the blinking component is removed by a signal processing technique in dealing with the brain wave signal, to analyze a brain wave activity. Accordingly, the blinking component superimposed on the brain wave signal has not been effectively utilized. If the blinking component superimposed on the brain wave signal is usable to detect blinking without using a device other than the electroencephalograph, it is possible to use the brain wave component and the blinking component without necessitating a device other than the electroencephalograph as a measurement instrument and to expect expansion of a use environment.

It is known that an indicator of the blinking component superimposed on the brain wave signal is different even on the same frontal region depending on a difference in measurement environment such as a position where the electroencephalograph is disposed. For example, in a case where the electroencephalograph is disposed at a position closer to a glabella, a blinking intensity increases. Further, the blinking intensity also changes depending on a line-of-sight direction of the user. When a device or an application is assumed that utilizes the brain wave component and the blinking component superimposed on the brain wave signal, a position of electrodes (measurement probes) of the electroencephalograph in contact with a head of the user may change depending on a mounted state or a mounting position of the device and a shape of the head of the user, to thereby change the blinking component superimposed on the brain wave signal. Occurrence of such a phenomenon causes a ratio between the brain wave component and the blinking component in measurement data to change between at the time of constructing a wakefulness level model and at the time of actual measurement, making it difficult to perform a correct emotion estimation. If it is possible to correct the indicator of the blinking component that varies in value between different pieces of the measurement data, a simple comparison is easily performed between the different pieces of the measurement data, making it possible to expand the scope of utilization of the blinking component superimposed on the brain wave signal.

Therefore, it is desired to develop a signal processing system that makes it possible to utilize, regardless of a difference in measurement environment, a blinking component superimposed on a brain wave signal for an emotion estimation.

### <1. First Embodiment>

### <1.1 Configuration>

FIG. 1 schematically illustrates a configuration example of a signal processing system according to a first embodiment of the present disclosure.

The signal processing system according to the first embodiment is a brain wave signal processing system that makes it possible to correct an indicator of a blinking component superimposed on a brain wave signal different in value between different pieces of measurement data in performing an emotion estimation using a brain wave component and a blinking component acquirable from an electroencephalograph.

The signal processing system according to the first embodiment includes a signal analysis unit 10, a blinking correction unit 20, and an emotion estimation unit 30.

The signal analysis unit 10 calculates a blinking component indicator and a brain wave signal feature amount on which the blinking component is superimposed, on the basis of the blinking component and the brain wave component included in the brain wave signal actually measured by the electroencephalograph of an electroencephalograph-mounted device 50. The signal analysis unit 10 includes a blinking detection section 11, a blinking component indicator calculation section 12, and a frequency analysis section 13.

The blinking correction unit 20 includes an actual-time correction data holding section 21 and a feature amount correction section 22. The feature amount correction section 22 is a correction section that corrects the brain wave signal feature amount on which the blinking component in the actually measured brain wave signal is superimposed, by comparing different pieces of the measurement data with each other in the brain wave signal and correcting the brain wave signal on the basis of a correction table generated on the basis of a brain wave signal serving as a reference for a correction. It is to be noted that the brain wave signal to be used to generate the correction table may be a signal based on an actual measurement result, or a signal based on a simulation result. The actual-time correction data holding section 21 holds a blinking component indicator serving as the reference for the correction and a brain wave signal feature amount on which a blinking component is superimposed and that serves as the reference for the correction. The blinking component indicator and the brain wave signal feature amount are calculated on the basis of the past actual measurement result of the brain wave signal serving as the reference for the correction or the simulation result of the brain wave signal serving as the reference for the correction.

The emotion estimation unit 30 includes an emotion estimation section 31. The emotion estimation section 31 performs the emotion estimation on the basis of the corrected brain wave signal feature amount on which the blinking component is superimposed.

It is to be noted that a main part of the signal processing system according to the first embodiment may include a computer including, for example, one or more central processing units (CPUs), one or more read-only memories (ROMs), and one or more random-access memories (RAMs). In this case, processing in each block in the signal processing system according to the first embodiment may be implemented when the one or more CPUs execute processing based on a program stored in the one or more ROMs or the one or more RAMs. Further, the processing in each block in the signal processing system according to the first embodiment may be implemented, for example, when the one or more CPUs execute processing based on a program supplied from the outside through a wired or wireless network.

### <1.2 Operation>

As an example, description is given of a flow of the correction to be performed in view of dependence of the electroencephalograph on its arrangement position when the blinking intensity serving as the blinking component indicator has changed with a difference in the arrangement position of the electroencephalograph. A flow of signal processing is as follows.

Process 1: The blinking detection section 11 detects an occurrence timing of the blinking component superimposed on the brain wave signal acquired by the electroencephalograph, and outputs the occurrence timing to the blinking component indicator calculation section 12.

Process 2: The blinking component indicator calculation section 12 calculates the blinking component indicator (e.g., the blinking intensity) using the brain wave signal on which the blinking component is superimposed and the blinking occurrence timing outputted from the blinking detection section 11, and outputs the blinking component indicator to the actual-time correction data holding section 21 and the feature amount correction section 22.

Process 3: The frequency analysis section 13 calculates various brain wave signal feature amounts (e.g., a power spectrum of a particular frequency band) on the basis of the brain wave signal on which the blinking component is superimposed, and outputs the various brain wave signal feature amounts to the actual-time correction data holding section 21 and the feature amount correction section 22.

Process 4: The actual-time correction data holding section 21 holds the calculated blinking component indicator and brain wave signal feature amounts.

Process 5: The feature amount correction section 22 compares, using the correction table, the different pieces of the measurement data with each other in distribution of the blinking component indicators and in distribution of the brain wave signal feature amounts on which the blinking components are superimposed. In a case where a trigger condition for execution of the correction is satisfied, the feature amount correction section 22 calculates a correction coefficient that corrects a difference in the distribution.

Process 6: The feature amount correction section 22 corrects, using the correction coefficient, a difference in the blinking component indicator and the brain wave signal feature amount on which the blinking component is superimposed between the different pieces of the measurement data, and outputs the corrected brain wave signal feature amount on which the blinking component is superimposed.

Process 7: The emotion estimation section 31 outputs an estimated emotion label on the basis of a model construction parameter of an emotion estimation model and the corrected brain wave signal feature amount.

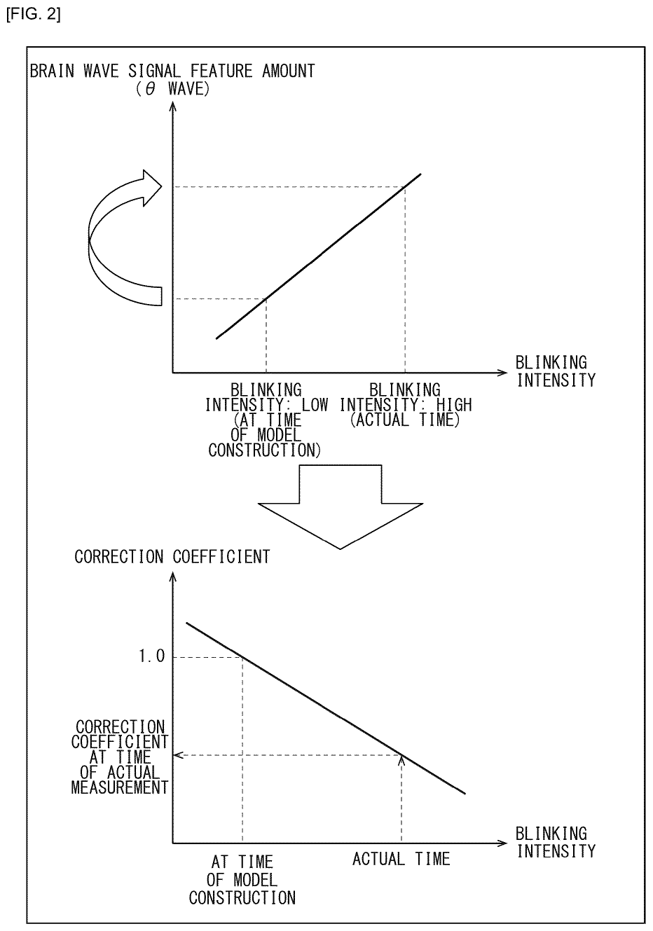

FIG. 2 illustrates an example of content of the correction table to be used for the correction to be performed by the blinking correction unit 20.

When the feature amount correction section 22 of the blinking correction unit 20 performs the correction in Process 5, a relation between the blinking component indicator and the brain wave signal feature amount on which the blinking component is superimposed is quantified in advance in the correction table on the basis of the simulation result or the past actual measurement result. For example, as illustrated in the upper part of FIG. 2, a relation between the blinking intensity and the brain wave signal feature amount (e.g., a θ-wave feature amount) is quantified. As illustrated in the lower part of FIG. 2, a correction coefficient that attenuates a difference in the blinking component indicator between the different pieces of the measurement data is calculable by comparing, using this correction table, the different pieces of the measurement data with each other in the distribution of the blinking intensities and in the distribution of the brain wave signal feature amounts on which the blinking intensities are superimposed. The blinking correction unit 20 holds, as the correction table, a table indicating the distribution of the blinking component indicators serving as the reference for the correction and the distribution of the brain wave signal feature amounts on which the blinking components are superimposed and that serve as the reference for the correction. The emotion estimation unit 30 performs the emotion estimation on the basis of the brain wave signal feature amount corrected on the basis of the correction coefficient.

### <Specific Example of Correction Method>

When the blinking correction unit 20 performs the correction in Process 5, the following two variations are conceivable regarding a data set to be corrected and a correction method.

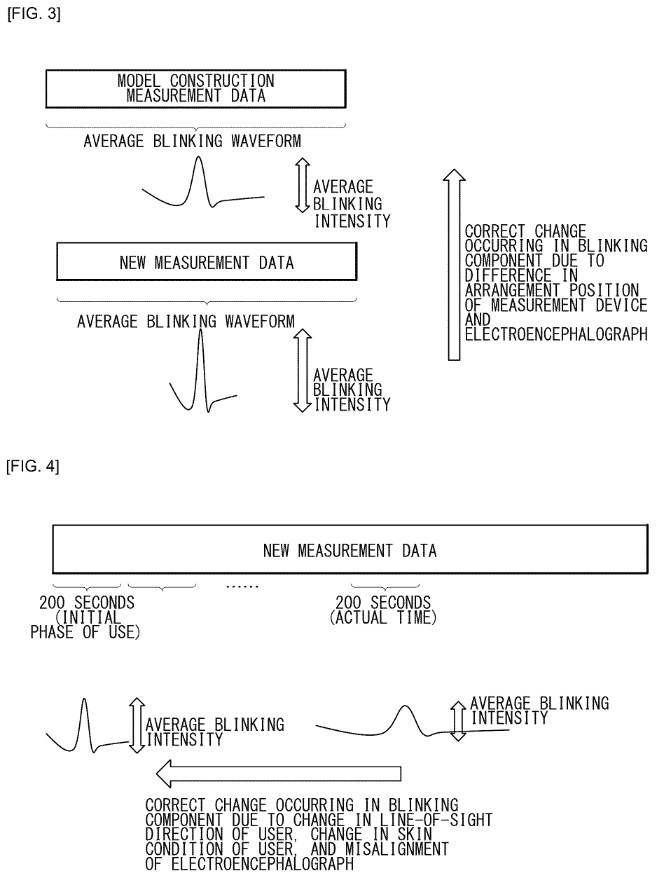

FIG. 3 illustrates a first example of the correction method to be performed by the blinking correction unit 20.

A data set (model construction measurement data) obtained in constructing the emotion estimation model in the past is defined as correction reference data, and a data set (new measurement data) newly acquired when the user uses the electroencephalograph-mounted device 50 and the application is corrected as correction target data.

In this method, the feature amount correction section 22 receives the blinking component indicator and the brain wave signal feature amount at the time of the model construction and the blinking component indicator and the brain wave signal feature amount calculated on the basis of the new measurement data serving as the correction target data, and corrects, using the correction table, the new measurement data to cause the new measurement data to match a state at the time of the model construction. This correction method enables the correction of a change occurring in the blinking component due to a difference in the electroencephalograph-mounted device 50 between at the time of the model construction and at the time of actual use and due to the difference caused in the arrangement position of the electroencephalograph with a change in its mounted state.

FIG. 4 illustrates a second example of the correction method to be performed by the blinking correction unit 20.

When the user uses the electroencephalograph-mounted device 50 and the application, a past data set acquired in an initial phase of use may be defined as the correction reference data, and a data set (in actual time) after a certain time period has elapsed since a start of the use may be corrected as the correction target data.

In this method, the actual-time correction data holding section 21 holds: the blinking component indicator that is obtained on the basis of the past actual measurement result (in the initial phase of the use) and serves as the correction reference data; and the brain wave signal feature amount on which the blinking component is superimposed, that is obtained on the basis of the past actual measurement result (in the initial phase of the use), and that serves as the correction reference data. Further, the feature amount correction section 22 receives the brain wave signal feature amount and the blinking component indicator held in the actual-time correction data holding section 21, the brain wave signal feature amount newly acquired and serving as the correction target data (in actual time), and the blinking component indicator serving as the correction target data. The feature amount correction section 22 compares the correction reference data with the correction target data using the correction table, and calculates, on the basis of a comparison result, the correction coefficient that corrects the difference in value of the blinking component indicator and the brain wave signal feature amount between respective pieces of data, i.e., the correction target data and the correction reference data. Accordingly, the feature amount correction section 22 corrects the difference in the blinking component indicator in real time in the measurement. This correction method enables the correction in real time of a change occurring in the blinking component in an actual use process with a change in the line-of-sight direction of the user and with a change in skin condition of the user.

### <Specific Example of Trigger for Execution of Correction>

When the blinking correction unit 20 performs the correction in Process 5, the following two variations are conceivable regarding a trigger for execution of the correction.

In a first example, the correction is controlled by setting a threshold. Specifically, the threshold is set for an evaluation value for the difference in the distribution of the blinking component indicators and the distribution of the brain wave signal feature amounts between the different pieces of the measurement data, and the correction is performed in a case where the difference in the distribution is greater than or equal to the threshold.

In a second example, data (e.g., an inertial measurement unit (IMU) sensor) on another modality (another piece of biological information) is used that is acquirable from a current function of the electroencephalograph-mounted device 50 (such as an earphone, a headset, or a head-mounted display). Specifically, the correction is performed in a case where an acceleration rate indicates a particular behavior or a value greater than or equal to a threshold.

### <1.3 Effects>

As described above, the signal processing system according to the first embodiment calculates the blinking component indicator and the brain wave signal feature amount on the basis of the blinking component and the brain wave component included in the brain wave signal actually measured by the electroencephalograph. Further, the brain wave signal feature amount on which the blinking component in the actually measured brain wave signal is superimposed is corrected on the basis of the correction table generated on the basis of the brain wave signal serving as the reference for the correction, and the emotion estimation is performed on the basis of the corrected brain wave signal feature amount on which the blinking component is superimposed. This makes it possible to utilize, regardless of a difference in measurement environment, the blinking component superimposed on the brain wave signal for the emotion estimation.

The signal processing system according to the first embodiment provides an example of a solution to a problem that a ratio between the blinking component and the brain wave component changes due to a different distribution of intensities of the blinking component superimposed on the brain wave signal, when the arrangement position of the electroencephalograph is different between the different pieces of the measurement data. This allows for performing the correction for each brain wave signal feature amount on which the blinking component is superimposed, without separating the brain wave component and the blinking component superimposed on the brain wave signal. This makes it possible to utilize, for the emotion estimation, the brain wave component and the blinking component of which the difference between the different pieces of the measurement data has been corrected, while reducing a computational cost. This provides a merit of a low difficulty in mounting (a wide range enabling mounting of) the signal processing system in the electroencephalograph-mounted device 50 and the application.

Further, the signal processing system according to the first embodiment makes it possible to robustly perform the emotion estimation in real time with the signal acquired by the electroencephalograph, even in an environment where a change in the position of the electroencephalograph due to the mounted state of the electroencephalograph-mounted device 50 or the shape of the head of the user occurs or where a change in the line-of-sight direction of the user occurs.

It is to be noted that the effects described herein are merely illustrative and not limiting, and other effects may be obtained. The same applies to effects of the following modification examples and other embodiments.

### 2. MODIFICATION EXAMPLES OF FIRST EMBODIMENT

Next, description is given of modification examples of the signal processing system according to the first embodiment. It is to be noted that, in the following, components substantially the same as those of the signal processing system according to the first embodiment described above are denoted by the same reference numerals to omit description thereof as appropriate.

### 2.1 Modification Example 1

FIG. 5 schematically illustrates a configuration example of a signal processing system according to Modification Example 1 of the first embodiment.

In the signal processing system (FIG. 1) according to the first embodiment described above, the example is indicated of performing the correction for each brain wave signal feature amount on which the blinking component is superimposed; however, the signal processing system according to Modification Example 1 corrects only the blinking component superimposed on the brain wave signal. The signal processing system according to Modification Example 1 includes a blinking and brain wave separation unit 14 provided in a stage preceding the signal analysis unit 10. The blinking and brain wave separation unit 14 first separates the brain wave component and the blinking component included in the brain wave signal. In the signal processing system according to Modification Example 1, the signal analysis unit 10 includes a brain wave analysis section 15 instead of the frequency analysis section 13.

The signal processing system according to Modification Example 1 has mainly the following six differences from the signal processing system (FIG. 1) according to the first embodiment described above.
1. The blinking and brain wave separation unit 14 separates the brain wave signal acquired by the electroencephalograph into the blinking component and the brain wave component.
2. The signal analysis unit 10 calculates the blinking component indicator and the brain wave signal feature amount on the basis of the blinking component and the brain wave component separated from the brain wave signal. The brain wave analysis section 15 calculates the brain wave signal feature amount on the basis of the brain wave signal from which the blinking component is removed, and outputs the brain wave signal feature amount to the emotion estimation section 31.
3. The actual-time correction data holding section 21 holds only the blinking component indicator calculated by the blinking component indicator calculation section 12.
4. The feature amount correction section 22 does not use the brain wave signal feature amount, and evaluates the difference in the distribution of only the blinking component indicators between the different pieces of the measurement data using the correction table. In a case where the trigger condition for execution of the correction is satisfied, the feature amount correction section 22 calculates the correction coefficient that corrects the difference in the distribution of the blinking component indicators.
5. The feature amount correction section 22 corrects the difference in the blinking component indicator between the different pieces of the measurement data using the correction coefficient, and outputs the corrected blinking component indicator.
6. The emotion estimation section 31 outputs the estimated emotion label on the basis of the model construction parameter of the emotion estimation model, the separated brain wave signal feature amount, and the corrected blinking component indicator.

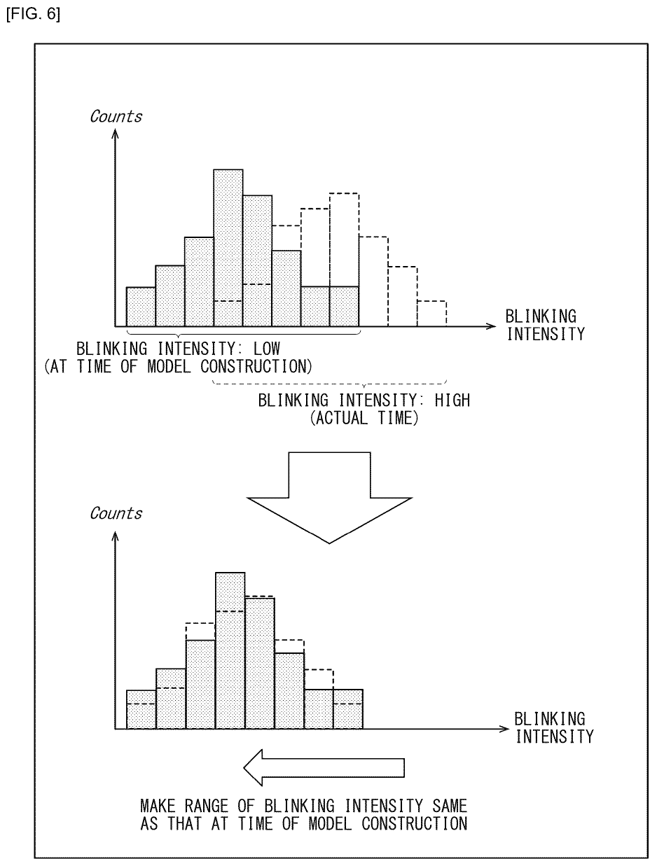

FIG. 6 illustrates an example of the correction method to be performed by the blinking correction unit 20 in the signal processing system according to Modification Example 1.

When the blinking correction unit 20 performs the correction, a relation of the blinking component indicator is quantified in advance in the correction table on the basis of the simulation result or the past actual measurement result. For example, as illustrated in the upper part of FIG. 6, the distribution of the blinking intensities between the different pieces of the measurement data is quantified. As illustrated in the lower part of FIG. 6, the correction coefficient that attenuates the difference in the blinking component indicator between the different pieces of the measurement data is calculable by comparing, using this correction table, the different pieces of the measurement data with each other in the distribution of the blinking intensities. The blinking correction unit 20 holds, as the correction table, a table indicating the distribution of the blinking component indicators serving as the reference for the correction.

The data set to be corrected, the correction method, and the trigger for execution of the correction when the blinking correction unit 20 performs the correction may be substantially similar to those of the signal processing system (FIG. 1) according to the first embodiment described above except that only the blinking component is to be corrected.

The signal processing system according to Modification Example 1 also provides an example of a solution to the problem that the ratio between the blinking component and the brain wave component changes due to the different distribution of the intensities of the blinking component superimposed on the brain wave signal. The signal processing system according to Modification Example 1 separates the brain wave component and the blinking component from the brain wave signal in an initial stage and deals with the separated brain wave component and blinking component individually. This provides a merit of enabling ensuring accuracy of each of the feature amounts, i.e., the brain wave component and the blinking component, and outputting an emotion estimation result with higher accuracy.

The signal processing system according to Modification Example 1 may have any other configuration, operation, and effects substantially similar to those of the signal processing system according to the first embodiment described above.

### 2.2 Modification Example 2

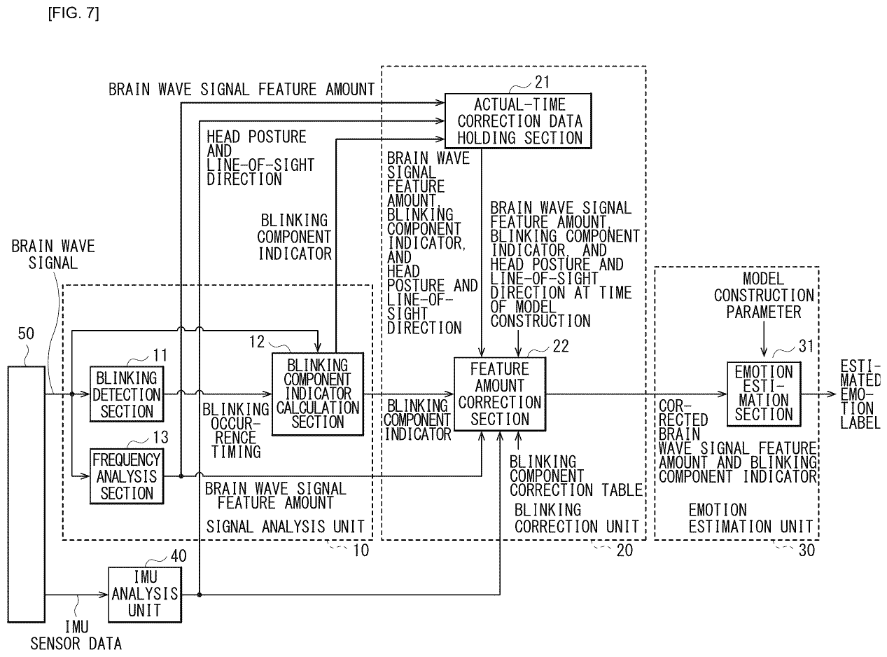

FIG. 7 schematically illustrates a configuration example of a signal processing system according to Modification Example 2 of the first embodiment.

In the signal processing system (FIG. 1) according to the first embodiment described above, data on another modality (another piece of biological information) acquirable from the current function of the electroencephalograph-mounted device 50 (such as the earphone, the headset, or the head-mounted display) may be used as additional information in the correction of the blinking component. The blinking correction unit 20 may correct the blinking component indicator or the brain wave signal feature amount on which the blinking component is superimposed, using, as the additional information, biological information that is different from the brain wave signal and outputted from the electroencephalograph-mounted device 50. It is to be noted that the biological information used herein includes, for example, physical information such as an acceleration rate or position information indicating a motion and a posture of the user. Such physical information is acquired from the electroencephalograph-mounted device 50.

FIG. 7 illustrates an example in which the electroencephalograph-mounted device 50 includes the IMU sensor, and sensor data from the IMU sensor is received by an IMU analysis unit 40. The signal processing system according to Modification Example 2 makes it possible to refine the correction of the blinking component by utilizing the data on the other modality acquirable from the current function of the electroencephalograph-mounted device 50 without adding another electroencephalograph or measurement function to the electroencephalograph-mounted device 50. The IMU analysis unit 40 makes it possible to grasp an approximate direction of a head posture and a line of sight with, for example, the IMU sensor mounted in the earphone serving as the electroencephalograph-mounted device 50. For example, the IMU analysis unit 40 outputs data on the head posture direction and the line-of-sight direction to the actual-time correction data holding section 21 and the feature amount correction section 22. The feature amount correction section 22 makes it possible to refine a calculation method of the correction coefficient by creating and referring to the correction table including information regarding the head posture direction and the line-of-sight direction, enabling an improvement in accuracy of the correction of the blinking component in a wider range of circumstances.

It is to be noted that, in the signal processing system (FIG. 5) according to Modification Example 1, the biological information different from the brain wave signal may be similarly used as the additional information to correct the blinking component indicator.

The signal processing system according to Modification Example 2 may have any other configuration, operation, and effects substantially similar to those of the signal processing system according to the first embodiment described above.

### 2.3 Modification Example 3

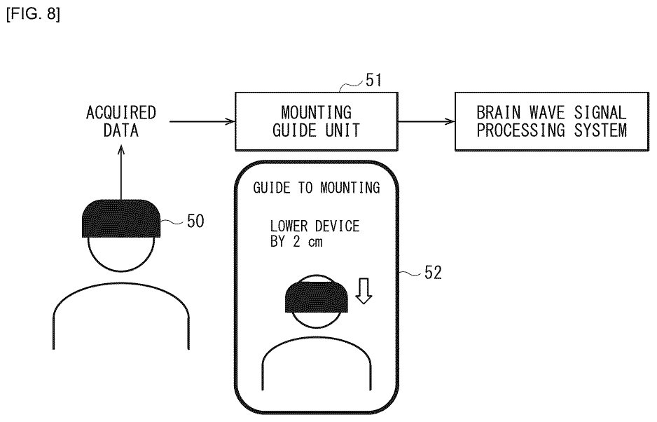

FIG. 8 schematically illustrates a configuration example of a signal processing system according to Modification Example 3.

As illustrated in FIG. 8, a mounting guide unit 51 may be provided as an assistance unit in a stage preceding any of the signal processing systems (FIGS. 1, 5, and 7) each described above as the brain wave signal processing system.

The mounting guide unit 51 estimates the mounted state of the electroencephalograph-mounted device 50 on the basis of the brain wave signal and IMU sensor data acquirable from the electroencephalograph-mounted device 50. The mounting guide unit 51 presents information that encourages an improvement in the mounted state on the basis of the estimated mounted state. For example, in a case where the mounted state is significantly different from an assumed mounted state, the mounting guide unit 51 may present, in advance on a user interface (UI) screen 52, the information that encourages the improvement in the mounted state. The mounting guide unit 51 provided in the stage preceding any of the signal processing systems described above makes it possible to improve an SN ratio of the signal in advance and assist in the correction to be performed on the blinking component by any of the signal processing systems described above.

The signal processing system according to Modification Example 3 uses, as the additional information in the correction of the blinking component, the data on the other modality acquirable from the current function of the electroencephalograph-mounted device 50. This makes it possible to grasp states of the electroencephalograph and the head in more detail and refine the correction of the blinking component.

The signal processing system according to Modification Example 3 may have any other configuration, operation, and effects substantially similar to those of the signal processing system according to the first embodiment described above.

### 2.4 Modification Example 4

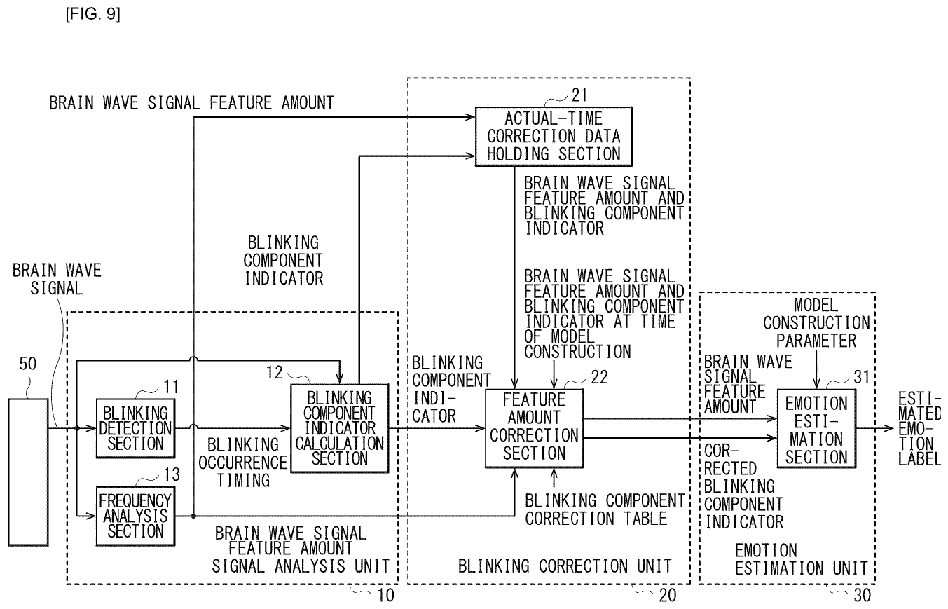

FIG. 9 schematically illustrates a configuration example of a signal processing system according to Modification Example 4 of the first embodiment.

In the signal processing system (FIG. 1) according to the first embodiment described above, the feature amount correction section 22 of the blinking correction unit 20 outputs, to the emotion estimation unit 30, the corrected brain wave signal feature amount on which the blinking component is superimposed without separating the blinking component from the brain wave signal; however, the feature amount correction section 22 may separate all or a portion of the blinking component from the brain wave signal feature amount.

The following two cases are conceivable regarding an output from the feature amount correction section 22 in the signal processing system according to Modification Example 4.

### <Case 1>

The feature amount correction section 22 corrects the brain wave signal feature amount on which a portion of the blinking component is superimposed and the blinking component indicator of the blinking component separated from the brain wave signal feature amount (not included in the brain wave signal feature amount), and outputs the corrected brain wave signal feature amount and blinking component indicator. The emotion estimation unit 30 performs the emotion estimation on the basis of the corrected blinking component indicator and the corrected brain wave signal feature amount on which the portion of the blinking component is superimposed.

### <Case 2>

The feature amount correction section 22 outputs the brain wave signal feature amount from which the entirety of the blinking component is separated and the blinking component indicator of the blinking component separated from the brain wave signal feature amount. The feature amount correction section 22 corrects the blinking component indicator obtained by separating the blinking component from the brain wave signal feature amount, and outputs the corrected blinking component indicator. The emotion estimation unit 30 performs the emotion estimation on the basis of the corrected blinking component indicator and the brain wave signal feature amount from which the entirety of the blinking component is separated.

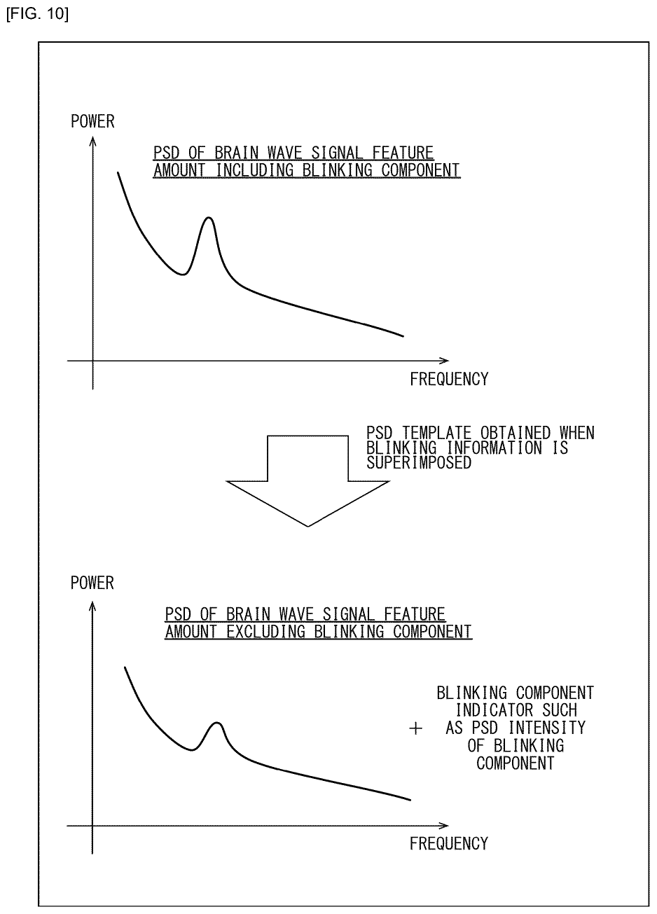

FIG. 10 illustrates an example of a method of separating the blinking component from the brain wave signal feature amount with the feature amount correction section 22.

In a case where the feature amount correction section 22 separates the blinking component from the brain wave signal feature amount as in Case 2, the separation may be performed in a frequency domain, as illustrated in FIG. 10, for example. FIG. 10 illustrates an example of a power spectral density (PSD) as an example of the brain wave signal feature amount in the frequency domain. The feature amount correction section 22 may separate the blinking component using, for example, a PSD template obtained when the blinking component is superimposed. In a case where the blinking and brain wave separation unit 14 is provided in an initial stage of the system as in, for example, the signal processing system (FIG. 5) according to Modification Example 1, the separation is performed in, for example, a time domain. This case, however, involves a computational cost. In a case where the separation is performed in the frequency domain in a subsequent stage of the system as in the signal processing system according to Modification Example 4, it is possible to improve reliability of the feature amounts, while suppressing the computational cost.

The signal processing system according to Modification Example 4 may have any other configuration, operation, and effects substantially similar to those of the signal processing system according to the first embodiment described above.

### <2.5 Modification Example 5>

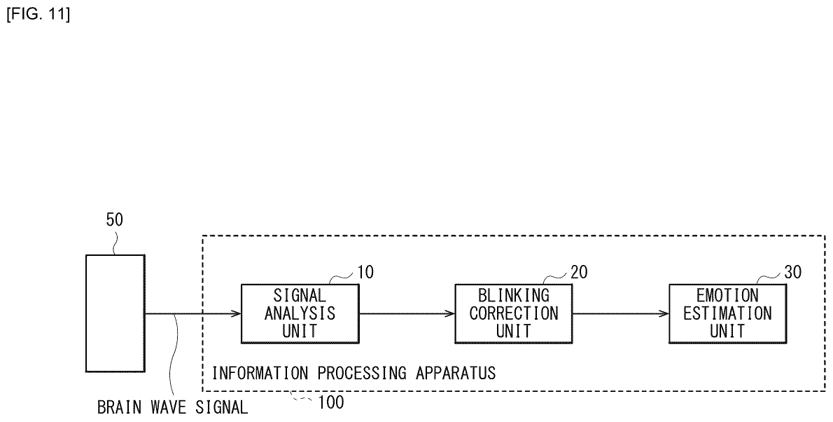

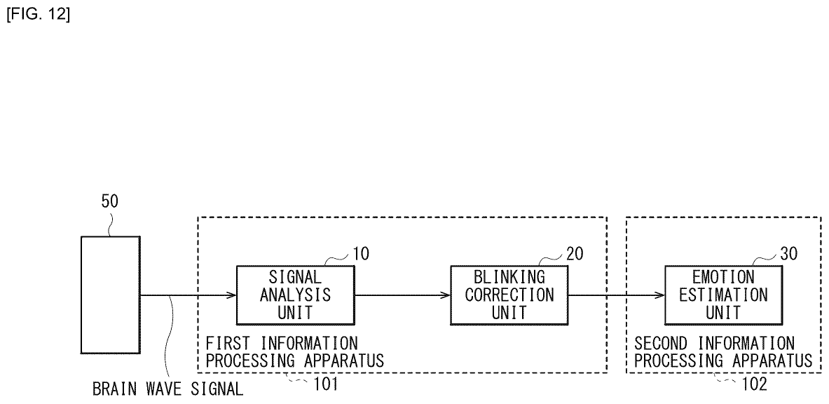

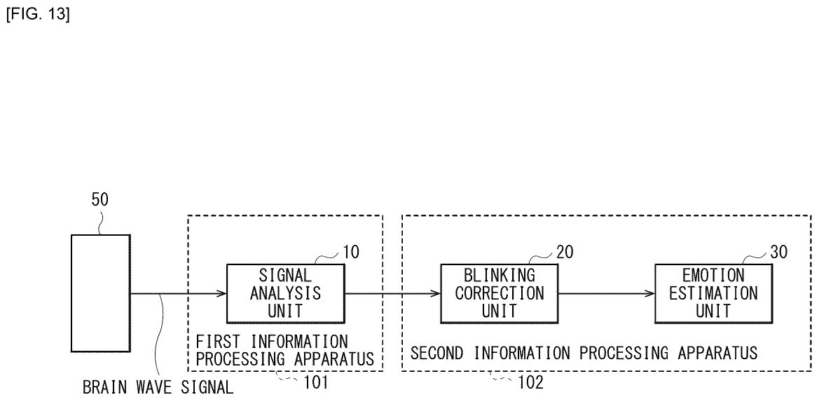

FIG. 11 schematically illustrates a first configuration example of a signal processing system according to Modification Example 5 of the first embodiment. FIG. 12 schematically illustrates a second configuration example of the signal processing system according to Modification Example 5. FIG. 13 schematically illustrates a third configuration example of the signal processing system according to Modification Example 5.

The respective configuration blocks (the signal analysis unit 10, the blinking correction unit 20, and the emotion estimation unit 30) in any of the signal processing systems described above may be configured by a single information processing apparatus 100, as illustrated in FIG. 11.

Further, the respective configuration blocks may be divided into a first information processing apparatus 101 and a second information processing apparatus 102. The second information processing apparatus 102 may perform the emotion estimation on the basis of a signal outputted from the first information processing apparatus 101. The first information processing apparatus 101 and the second information processing apparatus 102 may be communicably coupled to each other wirelessly or by wire. The first information processing apparatus 101 may be, for example, a personal computer or a compact device such as a smartphone. The second information processing apparatus 102 may be, for example, a server.

For example, the first information processing apparatus 101 may include the signal analysis unit 10 and the blinking correction unit 20, and the second information processing apparatus 102 may include the emotion estimation unit 30, as illustrated in FIG. 12.

Further, for example, the first information processing apparatus 101 may include the signal analysis unit 10, and the second information processing apparatus 102 may include the blinking correction unit 20 and the emotion estimation unit 30, as illustrated in FIG. 13.

The signal processing system according to Modification Example 5 may have any other configuration, operation, and effects substantially similar to those of the signal processing system according to the first embodiment described above.

### <3. Second Embodiment>

Next, description is given of a signal processing system according to a second embodiment of the present disclosure. It is to be noted that, in the following, components substantially the same as those of the signal processing system according to the first embodiment described above are denoted by the same reference numerals to omit description thereof as appropriate.

### <3.1 Overview>

### <Problem>

With an aim of utilization for evaluation and personalization of an application such as a game, an expectation for the emotion estimation using the blinking has been increased. Generally, there is found relevance between an emotion and the feature amount (the blinking component indicator) related to the blinking, such as a positive correlation between a blinking frequency and the wakefulness level. The emotion estimation may be performed on the basis of such a relation and a reference. Meanwhile, in some cases, the relation between the blinking and the emotion changes depending on a context such as a particular scene in the application such as the game. For example, the human has characteristics of blinking at a timing when a series of events ends. The blinking thus additionally occurs when the scene of the application is switched. Accordingly, when a frequency of scene switching is high, the number of times of blinking increases more than assumed because the number of times of blinking occurring at a timing of the scene switching is added to the number of times of blinking caused by how high or low the wakefulness level is. In such a scene, it is difficult to accurately estimate the wakefulness level with the blinking component indicator on the basis of the common reference.

PTL 2 (Japanese Unexamined Patent Application Publication No. 2009-261516) proposes a technique of switching model parameters of an emotion estimation model in accordance with a limited scene of a tension scene. However, there is not yet a specific technique that makes it possible to eliminate an influence of a difference in scene of an application and robustly perform the emotion estimation of an entire series of applications.

Regarding the emotion estimation using the blinking, the emotion estimation is performable with the same standard even in various scenes by correcting an influence of a context on a relationship between the blinking and the wakefulness level, leading to expansion to content such as the game of a scope of utilization of an emotion estimation technique using the blinking.

### <Basic Configuration>

The signal processing system according to the second embodiment is a blinking signal processing system that switches the correction method in a particular context (e.g., a particular genre, a particular scene, and particular elapsed time) in performing the emotion estimation using blinking information, to thereby achieve the emotion estimation with the same standard even in a context where a correlation between the blinking and the emotion changes from a reference relationship.

It is to be noted that the relationship between the blinking and the emotion (a blinking and emotion relationship) used herein refers to a relation between the blinking component indicator and the emotion (e.g., the wakefulness level). Representative examples of the blinking component indicator include, but are not limited to, the blinking frequency, the blinking intensity, and blinking entropy. An example of the relationship between the blinking and the emotion is a relationship in which, for example, the blinking frequency increases as the wakefulness level is higher.

The signal processing system according to the second embodiment has the following points.

In performing the emotion estimation with the blinking information, a reference context such as a scene is determined, and the relationship between the blinking and the emotion in the reference context is defined as a predetermined reference relationship. Further, in a case where it is detected on the basis of information regarding the content (e.g., metadata on the content and log data on the content) that the context has become a particular context where the relationship between the blinking and the emotion is different from the predetermined reference relationship, one or both of the following corrections (selections) are performed in accordance with the context. This reduces an error (e.g., minimizes the error) between the predetermined reference relationship and the relationship between the blinking and the emotion.
Correction of the blinking component indicator itself calculated with the blinking information.
Change in (selection of) a model parameter of the emotion estimation model using the blinking component indicator as an input.

It is to be noted that the signal processing system according to the second embodiment is applicable to the processing (the processing of the emotion estimation based on the blinking component indicator) to be performed by the emotion estimation unit 30 in any of the signal processing systems according to the first embodiment described above and the modification examples thereof.

### 3.2 SPECIFIC EXAMPLES

### Specific Example 1

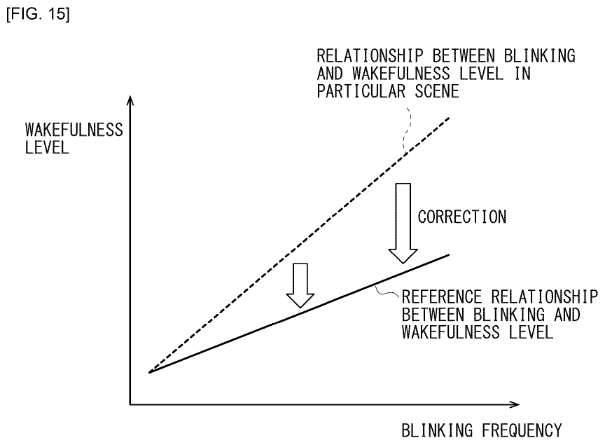

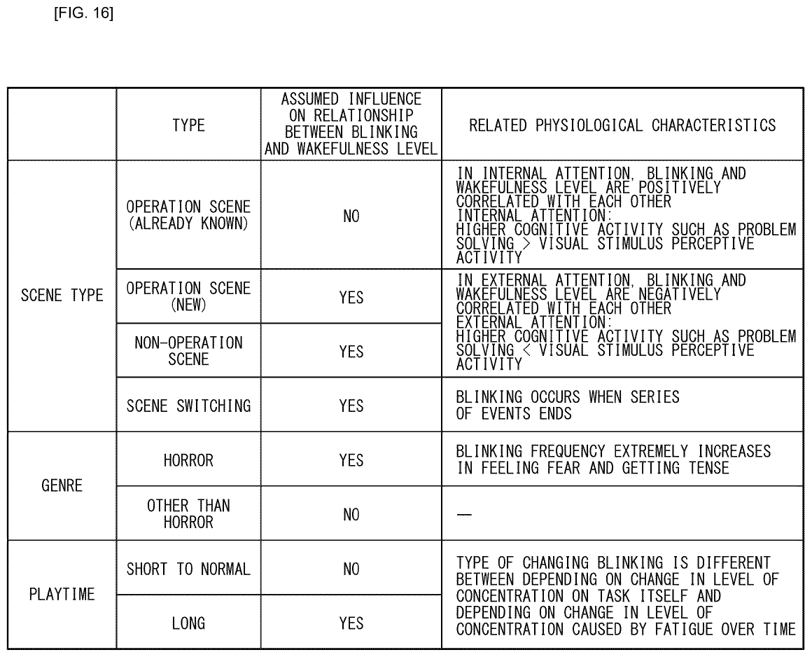

FIG. 14 is a block diagram schematically illustrating a configuration example (Specific Example 1) of the signal processing system according to the second embodiment. FIG. 15 is an explanatory graph schematically illustrating an example of the relationship between the blinking and the wakefulness level. FIG. 16 is an explanatory diagram schematically illustrating an example of the relationship between the blinking and the wakefulness level in each context.

The signal processing system according to Specific Example 1 includes a blinking emotion estimation unit 60 and a correction method selection unit (selection unit) 61. The blinking emotion estimation unit (emotion estimation unit) 60 includes a blinking component indicator calculation section 62 and a determiner 63.

The correction method selection unit 61 is a selection unit that selects, on the basis of the information regarding the content, the correction method of the emotion estimation in accordance with the context. In Specific Example 1, the correction method selection unit 61 selects, as the correction method of the emotion estimation, the model parameter of the emotion estimation model in accordance with the context. In a case where the context becomes a context having a state where the relationship between the blinking and the emotion is different from the predetermined reference relationship, the correction method selection unit 61 selects, as the correction method of the emotion estimation, a correction method that reduces the error (e.g., minimizes the error) with respect to the predetermined reference relationship.

The blinking emotion estimation unit 60 is an emotion estimation unit that performs the emotion estimation based on the blinking component indicator calculated on the basis of the blinking signal. The blinking emotion estimation unit 60 performs the emotion estimation based on the blinking component indicator through the correction method selected by the correction method selection unit 61. In Specific Example 1, the blinking emotion estimation unit 60 performs the emotion estimation using the model parameter selected by the correction method selection unit 61.

The information regarding the content includes at least one of the metadata on the content or the log data on the content. The context includes at least one of the genre of the content, the scene of the content, or the elapsed time of the content.

It is to be noted that a main part of the signal processing system according to the second embodiment may include a computer including, for example, one or more central processing units (CPUs), one or more read-only memories (ROMs), and one or more random-access memories (RAMs). In this case, processing in each block in the signal processing system according to the second embodiment may be implemented when the one or more CPUs execute processing based on a program stored in the one or more ROMs or the one or more RAMs. Further, the processing in each block in the signal processing system according to the second embodiment may be implemented, for example, when the one or more CPUs execute processing based on a program supplied from the outside through a wired or wireless network.

Here, as a specific example, description is given of an overview of the correction method to be performed when the blinking and emotion relationship has changed depending on a particular genre, a particular scene, and particular elapsed time of game content as the particular context. A flow of the signal processing is as follows.

Process 1: The blinking signal measured by, for example, an electrooculogram sensor is used as an input signal to the blinking component indicator calculation section 62, and the blinking component indicator calculation section 62 outputs the blinking component indicator.

Process 2: The metadata on the content and the log data on the content are used as input information regarding the content. In a case where any of the following states is detected, the correction method selection unit 61 selects a model parameter different from a normal one and outputs the selected model parameter. (1) A case where the content genre is horror, (2) a scene, such as gameplay in a new field or a movie scene, that causes a visual stimulus perceptive activity to be predominant over a higher cognitive activity, (3) a situation where frequent scene switching is performed, and (4) when a long time period has elapsed in the gameplay.

Process 3: The determiner 63 outputs the estimated emotion label using the blinking component indicator outputted in Process 1 and the model parameter outputted in Process 2.

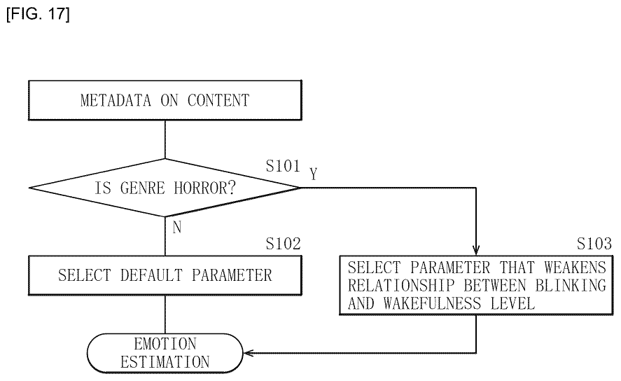

FIGS. 17 to 20, which are described later, illustrate a flow of detecting the respective states (1) to (4) and selecting an appropriate model parameter in Process 2. Here, as illustrated in FIG. 16, it is known that the relationship between the blinking and the wakefulness level is different depending on the particular genre, the particular scene, and the particular elapsed time of the game content. In a case where the particular genre, the particular scene, and the particular elapsed time are detected on the basis of the metadata on the content and the log data on the content, the signal processing system according to Specific Example 1 performs gain correction to allow for performing the emotion estimation with a unified standard and the reference relationship between the blinking and the wakefulness level, as illustrated in FIG. 15, by changing the model parameter. In FIG. 15, the blinking frequency is represented by a horizontal axis, and the wakefulness level is represented by a vertical axis.

### <Example of Content of Processing Using Genre Information as Trigger>

FIG. 17 is a flowchart illustrating an example of content of processing to be performed by the signal processing system according to Specific Example 1 using genre information as a trigger.

In a case where a scene that arouses fear is detected as the context, the correction method selection unit 61 selects, as the correction method of the emotion estimation, the correction method that reduces the error with respect to the predetermined reference relationship between the blinking and the wakefulness level. As illustrated in FIG. 16, it is known that the human has physiological characteristics of extremely increasing the blinking frequency when feeling fear and getting tense. For example, when a game genre is horror in the game, it is assumed that the blinking frequency constantly continues to increase more than usual and the estimated emotion label constantly has a “high wakefulness level”, making it unable to correctly estimate how high or low the wakefulness level is. Accordingly, in the signal processing system according to Specific Example 1, in a case where it is detected on the basis of the metadata on the game that the game genre is horror, the correction method selection unit 61 minimizes the error with respect to the predetermined reference relationship between the blinking and the wakefulness level in a horror genre by switching the model parameter to be used for the emotion estimation to a model parameter that weakens the relationship between the blinking and the wakefulness level.

First, the correction method selection unit 61 determines whether or not the game genre is horror (step S101). In a case where it is determined that the game genre is not horror (step S101: N), the correction method selection unit 61 thereafter selects a default model parameter as the model parameter to be used for the emotion estimation (step S102). Accordingly, in a case where the game genre is not horror, the blinking emotion estimation unit 60 performs the emotion estimation based on the default model parameter. In contrast, in a case where it is determined that the game genre is horror (step S101: Y), the correction method selection unit 61 thereafter selects, as the model parameter to be used for the emotion estimation, the model parameter that weakens the relationship between the blinking and the wakefulness level (step S103). Accordingly, in a case where the game genre is horror, the blinking emotion estimation unit 60 performs the emotion estimation based on the model parameter that weakens the relationship between the blinking and the wakefulness level.

### <Example of Content of Processing Using Scene Information as Trigger>

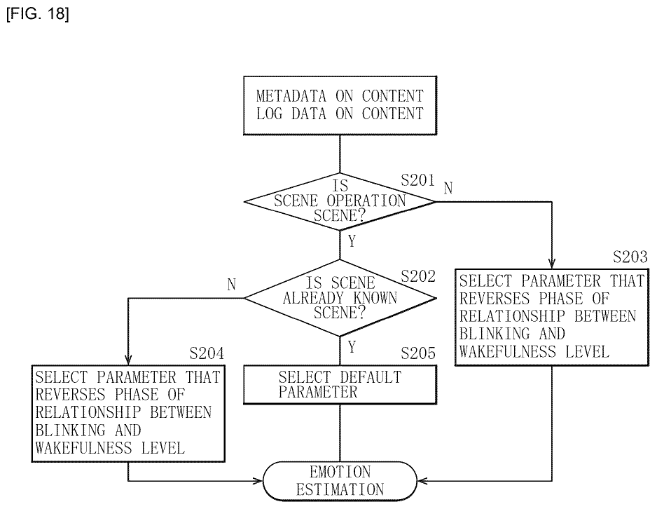

FIG. 18 is a flowchart illustrating an example of content of processing to be performed by the signal processing system according to Specific Example 1 using scene information as a trigger.

In a case where a scene that causes the perceptive activity with respect to a visual stimulus to be predominant is detected as the context, the correction method selection unit 61 selects, as the correction method of the emotion estimation, the correction method that reduces the error with respect to the predetermined reference relationship between the blinking and the wakefulness level. As illustrated in FIG. 16, it is known that the human has physiological characteristics in which the relationship between the blinking and the wakefulness level is negatively correlated in circumstances that cause the perceptive activity with respect to the visual stimulus to be predominant. In play in a field already known to a player in the game content, the visual stimulus perceptive activity is not predominant, and the relationship between the blinking and the wakefulness level is positively correlated. Meanwhile, in play in the field new to the player and a scene (such as the movie scene) not involving an operation, it is assumed that the relationship between the blinking and the wakefulness level is negatively correlated. Accordingly, the signal processing system according to Specific Example 1 determines whether or not the operation is performed and whether or not the scene is already presented to the player, on the basis of the metadata on the content and the log data on the content. Further, in a case where a new scene or a non-operation scene is detected, the signal processing system according to Specific Example 1 minimizes the error with respect to the predetermined reference relationship between the blinking and the wakefulness level by selecting a model parameter that reverses a phase of the relationship between the blinking and the wakefulness level.

First, the correction method selection unit 61 determines whether or not the scene involves the operation (step S201). In a case where it is determined that the scene does not involve the operation (step S201: N), the correction method selection unit 61 thereafter selects, as the model parameter to be used for the emotion estimation, the model parameter that reverses the phase of the relationship between the blinking and the wakefulness level (step S203). Accordingly, in a case of the non-operation scene, the blinking emotion estimation unit 60 performs the emotion estimation based on the model parameter that reverses the phase of the relationship between the blinking and the wakefulness level.

In contrast, in a case where it is determined that the scene involves the operation (step S201: Y), the correction method selection unit 61 thereafter determines whether or not the scene is already presented (step S202). In a case where it is determined that the scene is not already presented (step S202: N), the correction method selection unit 61 thereafter selects, as the model parameter to be used for the emotion estimation, the model parameter that reverses the phase of the relationship between the blinking and the wakefulness level (step S204). Accordingly, in a case of the new scene involving the operation, the blinking emotion estimation unit 60 performs the emotion estimation based on the model parameter that reverses the phase of the relationship between the blinking and the wakefulness level. In contrast, in a case where it is determined that the scene is already presented (step S202: Y), the correction method selection unit 61 thereafter selects the default model parameter as the model parameter to be used for the emotion estimation (step S205). Accordingly, in a case of the already presented scene involving the operation, the blinking emotion estimation unit 60 performs the emotion estimation based on the default model parameter.

### <Example of Content of Processing Using Scene Switching as Trigger>

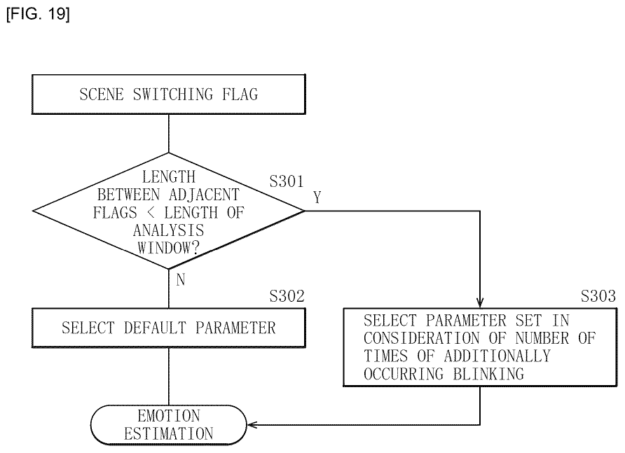

FIG. 19 is a flowchart illustrating an example of content of processing to be performed by the signal processing system according to Specific Example 1 using the scene switching as a trigger.

In a case where a situation where a plurality of times of the scene switching is included in an analysis window is detected as the context, the correction method selection unit 61 selects, as the correction method of the emotion estimation, the correction method that reduces the error with respect to the predetermined reference relationship between the blinking and the wakefulness level. As illustrated in FIG. 16, it is known that the human has physiological characteristics of blinking at a timing when a series of events ends. It is conceivable that, in the game, blinking not derived from the emotion additionally occurs at a timing when the scene switching is performed. The additional occurrence of the blinking is assumed to provide a disturbance factor to a change in the blinking frequency accompanied by a change in the wakefulness level, making a result of the estimation of the wakefulness level using the blinking inaccurate. Accordingly, the signal processing system according to Specific Example 1 uses, as the input information, a scene switching flag included in the log data on the content. In a case where it is determined that a length between adjacent flags, i.e., a scene length, is shorter than a length of the analysis window and that a situation where the scene switching is performed is included in the length of the analysis window, the signal processing system according to Specific Example 1 minimizes the error with respect to the predetermined reference relationship between the blinking and the wakefulness level by selecting a model parameter set in consideration of the number of times of the blinking additionally occurring at the time of the scene switching.

First, the correction method selection unit 61 determines whether or not the length between the adjacent flags is shorter than the length of the analysis window (step S301). In a case where it is determined that the length between the adjacent flags is not shorter than the length of the analysis window (step S301: N), the correction method selection unit 61 thereafter selects the default model parameter as the model parameter to be used for the emotion estimation (step S302). Accordingly, in a case where the situation where the scene switching is performed is not included in the length of the analysis window, the blinking emotion estimation unit 60 performs the emotion estimation based on the default model parameter. In contrast, in a case where it is determined that the length between the adjacent flags is shorter than the length of the analysis window (step S301: Y), the correction method selection unit 61 thereafter selects, as the model parameter to be used for the emotion estimation, the model parameter set in consideration of the number of times of the blinking additionally occurring at the time of the scene switching (step S303). Accordingly, in a case where the situation where the scene switching is performed is included in the length of the analysis window, the blinking emotion estimation unit 60 performs the emotion estimation based on the model parameter set in consideration of the number of times of the blinking additionally occurring at the time of the scene switching.

### <Example of Content of Processing Using Playtime as Trigger>

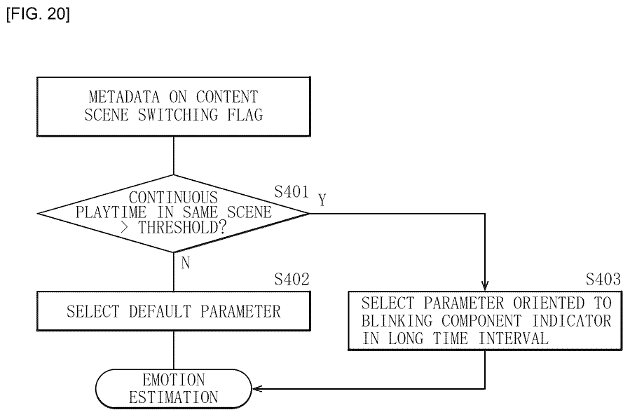

FIG. 20 is a flowchart illustrating an example of content of processing to be performed by the signal processing system according to Specific Example 1 using playtime as a trigger.

In a case where the same scene as the context lasts for a long time period exceeding a threshold, the correction method selection unit 61 selects, as the correction method of the emotion estimation, the correction method that reduces the error with respect to the predetermined reference relationship between the blinking and the wakefulness level. As illustrated in FIG. 16, it is known that the human has physiological characteristics in which a type of the changing blinking is different between depending on a change in the wakefulness level in working on a task itself and depending on a change in the wakefulness level caused by fatigue over time. When the fatigue occurs due to continuation of play in the same scene for a long time period in the game, the type of the changing blinking changes. If the emotion estimation is performed with a normal model parameter, it is assumed that the estimation result reflects a fatigue level different from an actual fatigue level. Accordingly, in a case where continuous playtime in the same scene exceeds the threshold in view of the log data on the content and the scene switching flag included in the log data, the signal processing system according to Specific Example 1 regards the play in the same scene as continuing for a long time period, and minimizes the error with respect to the predetermined reference relationship between the blinking and the wakefulness level by selecting a model parameter oriented to the blinking component indicator in a change over time.

First, the correction method selection unit 61 determines whether or not the continuous playtime in the same scene exceeds the threshold (step S401). In a case where it is determined that the continuous playtime in the same scene does not exceed the threshold (step S401: N), the correction method selection unit 61 thereafter selects the default model parameter as the model parameter to be used for the emotion estimation (step S402). Accordingly, in a case where the play in the same scene does not continue for a long time period, the blinking emotion estimation unit 60 performs the emotion estimation based on the default model parameter. In contrast, in a case where it is determined that the continuous playtime in the same scene exceeds the threshold (step S401: Y), the correction method selection unit 61 thereafter selects the model parameter oriented to the blinking component indicator in the change over time (a long time interval) (step S403). Accordingly, in a case where the play in the same scene continues for a long time period, the blinking emotion estimation unit 60 performs the emotion estimation based on the model parameter that is the model parameter oriented to the blinking component indicator in the change over time (the long time interval).

Specific Example 1 is an example of a solution to the problem that the accuracy of the estimation result decreases in performing the emotion estimation using the same standard as the reference relationship because the blinking and emotion relationship changes depending on the particular genre, the particular scene, and the particular elapsed time of the game content. In a case where the particular genre, the particular scene, and the particular elapsed time are detected, it is possible to perform the emotion estimation by the same determiner 63 with the same standard as the reference blinking and emotion relationship in the scene by selecting an appropriate model parameter. This provides a merit of expansion of the scope of availability of the emotion estimation using the blinking. Further, switching of the model parameters does not involve a high computational cost, thus providing a merit of a low difficulty in mounting the signal processing system in a device or an application.

### Specific Example 2

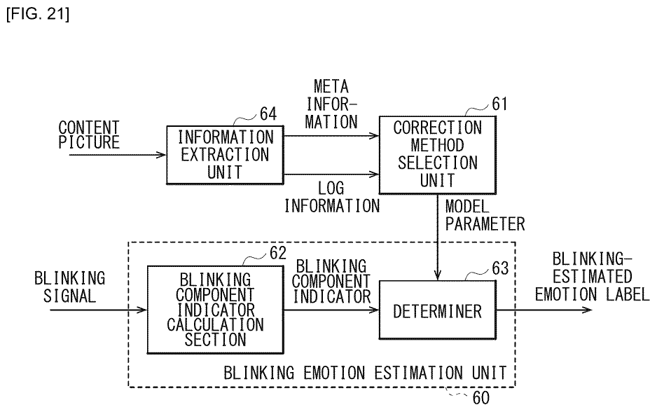

FIG. 21 is a block diagram schematically illustrating a configuration example (Specific Example 2) of the signal processing system according to the second embodiment.

The signal processing system according to Specific Example 2 has a configuration of further including an information extraction unit 64 in the signal processing system according to Specific Example 1.

The correction method selection unit 61 may not directly receive, as the input information, the metadata on the content and the log data on the content, and the information extraction unit 64 may be provided in a stage preceding the correction method selection unit 61. The information extraction unit 64 extracts log information and meta information through image processing using a content picture as the input information, and outputs the extracted log information and meta information to the correction method selection unit 61. The signal processing system according to Specific Example 2 makes it possible to acquire the log information and the meta information for detecting the particular genre, the particular scene, and the particular elapsed time in which the blinking and emotion relationship changes, even in a case where the metadata on the content and the log data on the content usable as the input information are not present.

### Specific Example 3

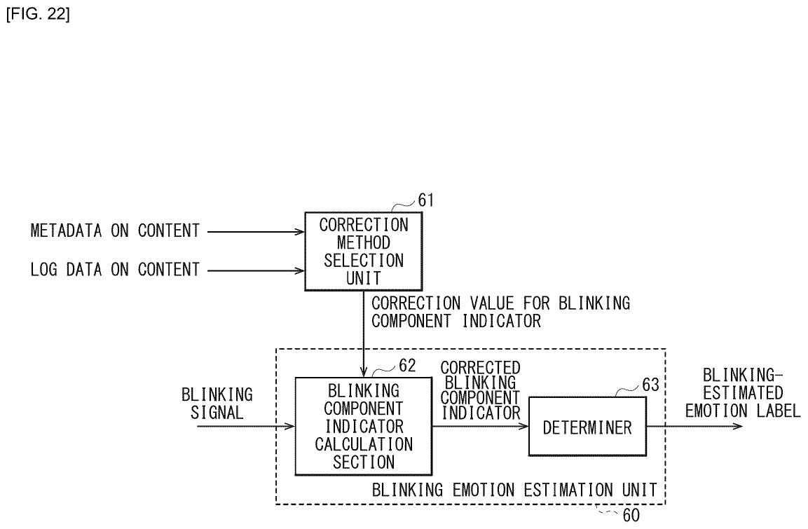

FIG. 22 is a block diagram schematically illustrating a configuration example (Specific Example 4) of the signal processing system according to the second embodiment.

As the correction method of the emotion estimation, the correction method selection unit 61 may calculate a correction value for the blinking component indicator in accordance with the context, instead of selecting the model parameter of the emotion estimation model in accordance with the context. The blinking emotion estimation unit 60 may perform the emotion estimation based on the blinking component indicator corrected using the correction value for the blinking component indicator calculated by the correction method selection unit 61. In the signal processing system according to Specific Example 3, in a case where the particular genre, the particular scene, and the particular elapsed time are detected in a flow similar to that of Specific Example 1 on the basis of the metadata on the content and the log data on the content, the blinking component indicator calculation section 62 corrects the value of the blinking component indicator. This allows the determiner 63 to perform the emotion estimation with the same standard as that in a case where the blinking component indicator in a normal genre, scene, and elapsed time is used. An example of the correction method is as follows. When the situation where the scene switching is performed appears frequently in the content, the blinking additionally occurs in the situation where the switching is performed. Assuming that the number of situations where the scene switching is performed and that are included in the analysis window is denoted by n and the blinking component indicator related to an original blinking frequency is denoted by b_origin, a corrected blinking component indicator b_updated is calculated by the following expression. It is to be noted that param refers to a correction value determined on the basis of a relationship between the blinking component indicator and a frequency of appearances of the situations where the scene switching is performed. This correction method is a mere example, and a specific correction method is not limited thereto.

b_updated=b_origin−(param×N)

The signal processing system according to Specific Example 3 makes it possible to perform the correction and the emotion estimation with higher accuracy by correcting the blinking component indicator instead of the model parameter. This further provides a merit of utilizing this correction method as it is, even when the determiner 63 itself is updated or the determiner 63 to be used is changed.

### Specific Example 4

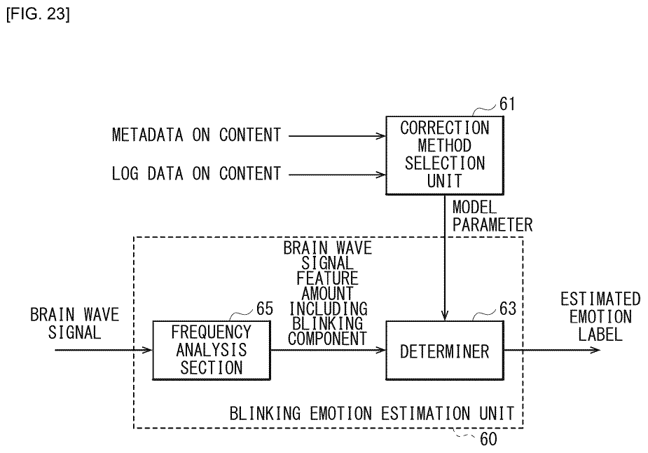

FIG. 23 is a block diagram schematically illustrating a configuration example (Specific Example 4) of the signal processing system according to the second embodiment.

The signal processing system according to Specific Example 4 has a configuration of including a frequency analysis section 65 instead of the blinking component indicator calculation section 62 in the signal processing system according to Specific Example 1. The frequency analysis section 65 calculates, on the basis of the brain wave signal, the brain wave signal feature amount including the blinking component, and outputs, to the determiner 63, the calculated brain wave signal feature amount including the blinking component.

In the signal processing system according to Specific Example 1, only the blinking signal measurable by the electrooculogram sensor or the like is to be corrected; however, the electroencephalograph may measure the brain wave signal including the blinking component, and the brain wave signal including the blinking component may be corrected, similarly to the signal processing system according to the first embodiment described above. As described above in Specific Example 1, what is influenced by the context such as the particular scene is mainly the blinking component indicator. Meanwhile, in a case where the brain wave signal is acquired from the electroencephalograph, the blinking component is superimposed on the brain wave signal. The method of utilizing this blinking component as one piece of information has been described in the first embodiment described above. In a case where the blinking and emotion relationship changes depending on the particular genre, the particular scene, and the particular elapsed time, a relation between the emotion and a feature amount derived from the brain wave signal including the blinking component also changes. The correction, similar to Specific Example 1, of the brain wave signal feature amount including the blinking component makes it possible to minimize the error between the predetermined reference relationship and the relation between the emotion and the feature amount derived from the brain wave signal including the blinking component in the particular genre, the particular scene, and the particular elapsed time, and perform the emotion estimation with the same standard as the predetermined reference relationship. In Specific Example 4, it is necessary to measure the brain wave signal having an information amount necessary also as the brain wave, while clearly superimposing the blinking component having an information amount necessary for the correction. Accordingly, in implementing Specific Example 4, it is conceivable to position, in the frontal area relatively close to an eyebrow, electrodes integrated in a device.

The signal processing system according to Specific Example 4 makes it possible to utilize, for the emotion estimation, the brain wave component and the blinking component included in the brain wave signal acquired from a single sensor, i.e., the electroencephalograph. Accordingly, higher accuracy of the emotion estimation is expectable.

### Specific Example 5

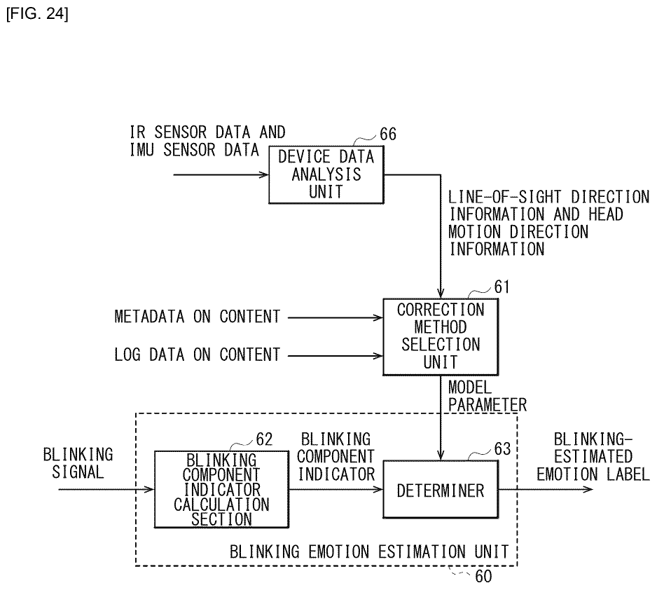

FIG. 24 is a block diagram schematically illustrating a configuration example (Specific Example 5) of the signal processing system according to the second embodiment.

The signal processing system according to Specific Example 5 has a configuration of further including a device data analysis unit 66 in the signal processing system according to Specific Example 1.

In the configuration of the signal processing system according to Specific Example 1, data on another modality acquirable from the current function of a device (such as the head-mounted display or a game machine) mounted with a sensor that acquires the blinking signal may be used as the additional information when the correction method selection unit 61 selects a correction method. The signal processing system according to Specific Example 5 makes it possible to achieve higher accuracy of the correction of the blinking and emotion relationship in the particular genre, the particular scene, and the particular elapsed time by utilizing, via the device data analysis unit 66, the data on the other modality acquirable from the current function of the device mounted with a sensor (e.g., the electrooculogram sensor) that measures the blinking, without adding a new sensor or measurement function. For example, an IR camera or an acceleration rate sensor is occasionally already incorporated in the game machine or the head-mounted display, and enables acquisition of the line-of-sight direction or the head posture direction. Approximately presuming an object to which the user pays attention using the head posture direction and the line-of-sight direction makes it possible to identify whether or not the genre, the scene, and the elapsed time are the particular genre, the particular scene, and the particular elapsed time, refine a calculation method of the correction value for the blinking and emotion relationship, and improve accuracy of the correction of the blinking and emotion relationship in the wider range of circumstances.

### Specific Example 6

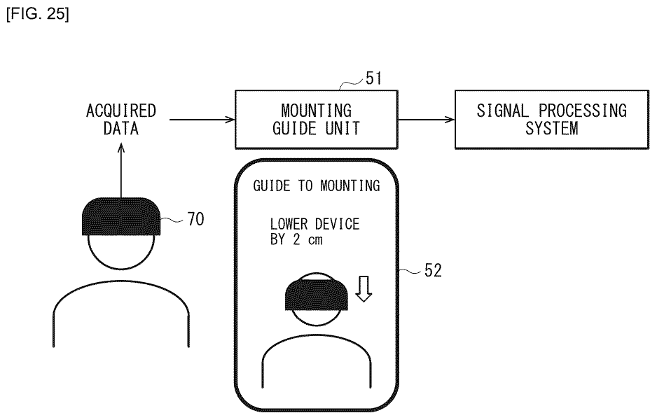

FIG. 25 is a block diagram schematically illustrating a configuration example (Specific Example 6) of the signal processing system according to the second embodiment.

As illustrated in FIG. 25, the mounting guide unit 51 may be provided as an assistance unit in a stage preceding any of the signal processing systems according to the respective specific examples described above.

The mounting guide unit 51 estimates, on the basis of a blinking signal and IMU sensor data acquirable from a device 70 mounted with the electrooculogram sensor or the electroencephalograph, a mounted state of the device 70. The mounting guide unit 51 presents information that encourages an improvement in the mounted state on the basis of the estimated mounted state. For example, in a case where the mounted state is significantly different from an assumed mounted state, the mounting guide unit 51 presents, in advance on the user interface (UI) screen 52, the information that encourages the improvement in the mounted state. The mounting guide unit 51 provided in the stage preceding any of the signal processing systems according to the respective specific examples makes it possible to improve the SN ratio of the signal in advance and assist in correction processing to be performed by the correction method selection unit 61 of any of the signal processing systems according to the respective specific examples.

### Specific Example 7

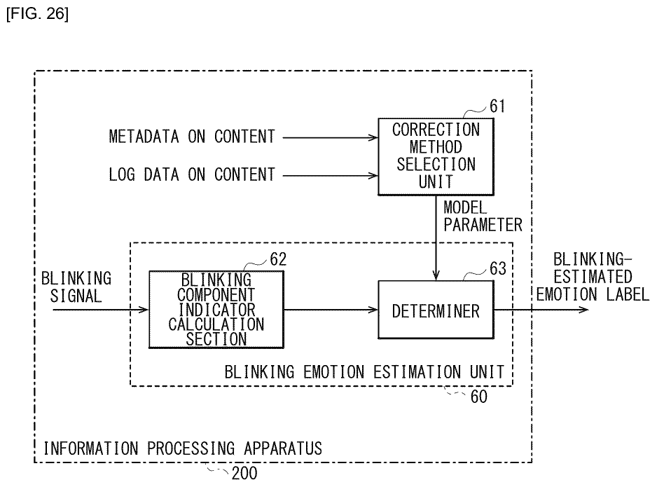

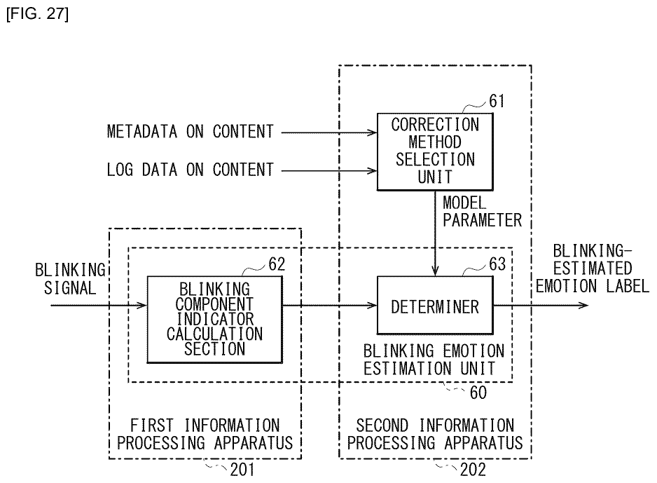

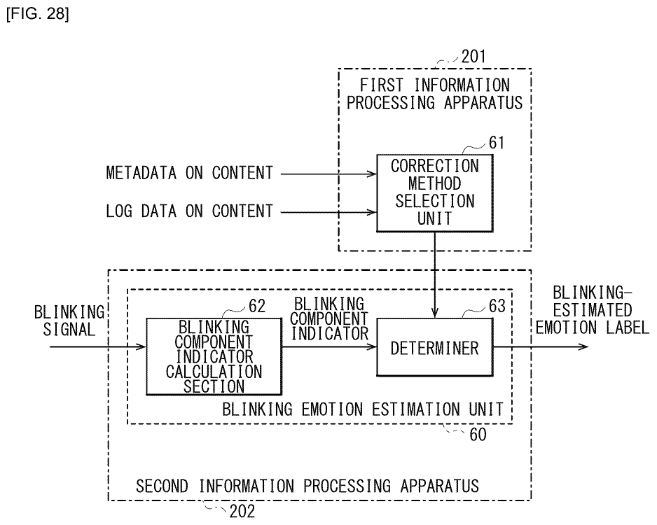

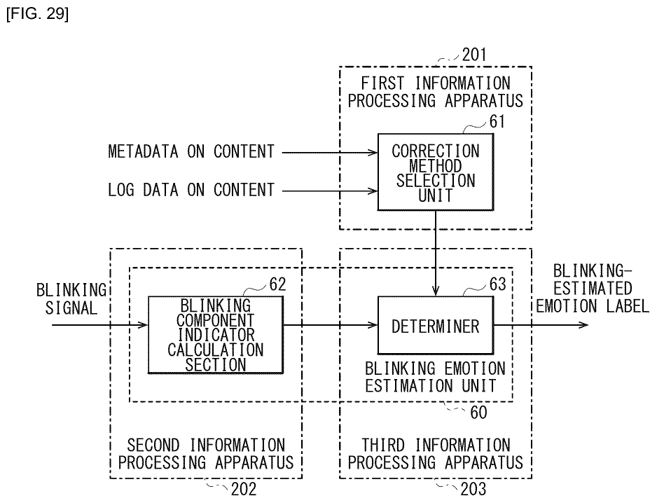

FIGS. 26 to 29 are each a block diagram schematically illustrating a configuration example (Specific Example 7) of the signal processing system according to the second embodiment. FIG. 26 illustrates a first configuration example of Specific Example 7. FIG. 27 illustrates a second configuration example of Specific Example 7. FIG. 28 illustrates a third configuration example of Specific Example 7. FIG. 29 illustrates a fourth configuration example of Specific Example 7.

Any of the signal processing systems according to the respective specific examples may be configured by a single information processing apparatus 200, as in the configuration example of FIG. 26. Further, the respective configuration blocks may be divided into a first information processing apparatus 201 and a second information processing apparatus 202, as in the configuration example of FIG. 27. The first information processing apparatus 201 may include the blinking component indicator calculation section 62. The second information processing apparatus 202 may include the correction method selection unit 61 and the determiner 63 that each receive, as an input signal, a signal outputted from the first information processing apparatus 201. The first information processing apparatus 201 and the second information processing apparatus 202 may be coupled to each other wirelessly or by wire. As a specific example of the apparatus, the first information processing apparatus 201 is a personal computer or a compact device such as a smartphone or a device linked to a content application such as the game machine, and the second information processing apparatus 202 is a server or the like.

Furthermore, the respective configuration blocks may be divided into the first information processing apparatus 201 including the correction method selection unit 61, and the second information processing apparatus 202 including the blinking component indicator calculation section 62 and the determiner 63, as in the configuration example of FIG. 28. Furthermore, the respective configuration blocks may be divided into the first information processing apparatus 201 including the correction method selection unit 61, the second information processing apparatus 202 including the blinking component indicator calculation section 62, and a third information processing apparatus 203 including the determiner 63, as in the configuration example of FIG. 29.

### <3.3 Effects>

As described above, the signal processing system according to the second embodiment selects, on the basis of the information regarding the content, the correction method of the emotion estimation in accordance with the context, and performs the emotion estimation based on the blinking component indicator through the selected correction method. This enables the emotion estimation in consideration of the influence of the context.

In experiencing the content, the signal processing system according to the second embodiment determines the reference situation having the genre, the scene, and the elapsed time, and defines the blinking and emotion relationship in the reference situation as the predetermined reference relationship. When the particular genre, the particular scene, and the particular elapsed time in which the blinking and emotion relationship changes are detected on the basis of the meta information and the log information regarding the content, the signal processing system according to the second embodiment switches the correction methods, and minimizes the error between the blinking and emotion relationship and the predetermined reference relationship. This makes it possible to perform the emotion estimation with the same determiner and the same standard as the blinking and emotion relationship in the reference situation, leading to a robust emotion estimation using the blinking and expansion of the scope of availability of the emotion estimation using the blinking. Further, switching of the model parameters does not involve a high computational cost, allowing for a low difficulty in mounting the signal processing system in a device or an application.

Further, the signal processing system according to the second embodiment may extract the log information and the meta information regarding the content from the content picture, and select the correction method using the extracted information. This makes it possible to select an appropriate correction method using the information related to the content, even if the meta information and the log information regarding the content are not present or are unable to be outputted to the outside.

Furthermore, the signal processing system according to the second embodiment may perform the correction for each brain wave signal feature amount including the blinking component. Accordingly, because the brain wave component and the blinking component are acquired and corrected without being separated, it is possible to utilize, for the emotion estimation, two types of information (brain wave information and blinking information) of which a deviation from a predetermined reference relationship is minimized, while reducing the computational cost. This leads to the higher accuracy of the emotion estimation.

Furthermore, the signal processing system according to the second embodiment may use, as the additional information in the correction, the data on the other modality acquirable from the current function of the device mounted with the sensor that acquires the blinking signal. This makes it possible to grasp states of the sensor and the head in more detail, and refine the correction method.

### 4. Other Embodiments>

A technique according to the present disclosure is not limited to the description of each of the above-described embodiments, and various modifications are possible.

For example, the present technology may have the following configurations.

According to the present technology having the following configurations, a blinking component indicator and a brain wave signal feature amount are calculated on the basis of a blinking component and a brain wave component included in a brain wave signal actually measured by an electroencephalograph. Further, a correction of the blinking component indicator or the brain wave signal feature amount on which the blinking component is superimposed in the actually measured brain wave signal is performed on the basis of a correction table generated on the basis of a brain wave signal serving as a reference for the correction. An emotion estimation is performed on the basis of at least the corrected blinking component indicator or the corrected brain wave signal feature amount on which the blinking component is superimposed.

This makes it possible to utilize, regardless of a difference in measurement environment, the blinking component superimposed on the brain wave signal for the emotion estimation.

Further, according to the present technology, a correction method of the emotion estimation in accordance with a context is selected on the basis of information regarding content, and the emotion estimation based on the blinking component indicator is performed through the selected correction method.

This makes it possible to perform the emotion estimation in consideration of an influence of the context.

(1)

A signal processing system including:
a signal analysis unit that calculates a blinking component indicator and a brain wave signal feature amount on a basis of a blinking component and a brain wave component included in a brain wave signal actually measured by an electroencephalograph;
a correction unit that performs a correction of the blinking component indicator or the brain wave signal feature amount in the actually measured brain wave signal on a basis of a correction table generated on a basis of a brain wave signal serving as a reference for the correction, the blinking component being superimposed on the brain wave signal feature amount; and
an emotion estimation unit that performs an emotion estimation on a basis of at least the corrected blinking component indicator or the corrected brain wave signal feature amount on which the blinking component is superimposed.
   (2)

The signal processing system according to (1), in which
the signal analysis unit calculates the blinking component indicator and the brain wave signal feature amount on which the blinking component is superimposed,
the correction unit corrects the brain wave signal feature amount on which the blinking component is superimposed, and
the emotion estimation unit performs the emotion estimation on a basis of the corrected brain wave signal feature amount on which the blinking component is superimposed.
   (3)

The signal processing system according to (1), in which
the signal analysis unit calculates the blinking component indicator and the brain wave signal feature amount on a basis of the blinking component and the brain wave component that are separated from the actually measured brain wave signal,
the correction unit performs the correction of the blinking component indicator, and
the emotion estimation unit performs the emotion estimation on a basis of the separated brain wave signal feature amount and the corrected blinking component indicator.
   (4)

The signal processing system according to (1), in which
the signal analysis unit calculates the blinking component indicator and the brain wave signal feature amount on which the blinking component is superimposed,
the correction unit
   separates the blinking component from the brain wave signal feature amount on which the blinking component is superimposed, and
   performs the correction of the blinking component indicator of the separated blinking component, and
the emotion estimation unit performs the emotion estimation on a basis of the separated brain wave signal feature amount and the corrected blinking component indicator.
   (5)

The signal processing system according to (1), in which
the signal analysis unit calculates the blinking component indicator and the brain wave signal feature amount on which the blinking component is superimposed,
the correction unit
   separates a portion of the blinking component from the brain wave signal feature amount on which the blinking component is superimposed, and
   performs the correction of the brain wave signal feature amount on which a portion of the blinking component is superimposed and the blinking component indicator of the separated blinking component, and
the emotion estimation unit performs the emotion estimation on a basis of the corrected blinking component indicator and the corrected brain wave signal feature amount on which the portion of the blinking component is superimposed.
   (6)

The signal processing system according to any one of (1) to (5), in which the correction unit holds, out of the blinking component indicator serving as the reference for the correction and the brain wave signal feature amount on which the blinking component is superimposed and that serves as the reference for the correction, at least the blinking component indicator serving as the reference for the correction, the blinking component indicator and the brain wave signal feature amount being each calculated on a basis of a past actual measurement result of the brain wave signal serving as the reference for the correction or a simulation result of the brain wave signal serving as the reference for the correction.

(7)

The signal processing system according to any one of (1) to (6), in which the correction unit holds, as the correction table, a table indicating: a distribution of the blinking component indicator serving as the reference for the correction; or a distribution of the brain wave signal feature amount on which the blinking component is superimposed and that serves as the reference for the correction.

(8)

The signal processing system according to any one of (1) to (7), in which the correction unit
performs, using the correction table, a comparison between: the blinking component indicator serving as correction target data or the brain wave signal feature amount on which the blinking component is superimposed and that serves as the correction target data; and the blinking component indicator obtained on a basis of a past actual measurement result and serving as correction reference data or the brain wave signal feature amount on which the blinking component is superimposed, that is obtained on a basis of the past actual measurement result, and that serves as the correction reference data, and
calculates, on a basis of a result of the comparison, a correction coefficient that corrects a difference in a value of the blinking component indicator or the brain wave signal feature amount between pieces of data that are the correction target data and the correction reference data.
   (9)

The signal processing system according to (8), in which the emotion estimation unit performs the emotion estimation on a basis of at least: the blinking component indicator corrected on a basis of the correction coefficient; or the brain wave signal feature amount corrected on a basis of the correction coefficient.

(10)

The signal processing system according to any one of (1) to (9), in which the correction unit performs the correction of the blinking component indicator or the brain wave signal feature amount using, as additional information, biological information different from the brain wave signal outputted from a device mounted with the electroencephalograph, the blinking component being superimposed on the brain wave signal feature amount.

(11)

The signal processing system according to any one of (1) to (10), further including a mounting guide unit that estimates a mounted state of a device mounted with the electroencephalograph, the mounting guide unit presenting information that encourages an improvement in the mounted state on a basis of the estimated mounted state.

(12)

The signal processing system according to any one of (1) to (11), further including a selection unit that selects a correction method of the emotion estimation on a basis of information regarding content, the correction method being in accordance with a context, in which
the emotion estimation unit performs the emotion estimation based on the blinking component indicator through the correction method selected by the selection unit.
   (13)

The signal processing system according to (12), in which
the information regarding the content includes at least one of metadata on the content or log data on the content, and
the context includes at least one of a genre of the content, a scene of the content, or elapsed time of the content.
   (14)

The signal processing system according to (12) or (13), in which
as the correction method of the emotion estimation, the selection unit selects a model parameter of an emotion estimation model in accordance with the context, or calculates a correction value for the blinking component indicator in accordance with the context, and
the emotion estimation unit performs the emotion estimation using the model parameter selected by the selection unit, or performs the emotion estimation based on the blinking component indicator corrected using the correction value for the blinking component indicator calculated by the selection unit.
   (15)

The signal processing system according to any one of (12) to (14), in which the selection unit selects, as the correction method of the emotion estimation, a correction method that reduces an error with respect to a predetermined reference relationship, in a case where the context becomes a context having a state where a relationship between blinking and an emotion is different from the predetermined reference relationship.

(16)

The signal processing system according to (15), in which the selection unit selects, as the correction method of the emotion estimation, the correction method that reduces the error with respect to the predetermined reference relationship, in a case where a scene that causes a perceptive activity with respect to a visual stimulus to be predominant is detected as the context.

(17)

The signal processing system according to (15) or (16), in which the selection unit selects, as the correction method of the emotion estimation, the correction method that reduces the error with respect to the predetermined reference relationship, in a case where a situation where a plurality of times of scene switching is included in an analysis window is detected as the context.

(18)

The signal processing system according to any one of (15) to (17), in which the selection unit selects, as the correction method of the emotion estimation, the correction method that reduces the error with respect to the predetermined reference relationship, in a case where a scene that arouses fear is detected as the context.

(19)

The signal processing system according to any one of (15) to (18), in which the selection unit selects, as the correction method of the emotion estimation, the correction method that reduces the error with respect to the predetermined reference relationship, in a case where a same scene as the context lasts for a long time period exceeding a threshold.

(20)

A signal processing system including a second information processing apparatus that performs an emotion estimation on a basis of a signal outputted from a first information processing apparatus,
the first information processing apparatus including
   a signal analysis unit that calculates a blinking component indicator and a brain wave signal feature amount on a basis of a blinking component and a brain wave component included in a brain wave signal actually measured by an electroencephalograph, and
   a correction unit that performs a correction of the blinking component indicator or the brain wave signal feature amount in the actually measured brain wave signal on a basis of a correction table generated on a basis of a brain wave signal serving as a reference for the correction, the blinking component being superimposed on the brain wave signal feature amount, and
the second information processing apparatus including an emotion estimation unit that performs the emotion estimation on a basis of at least the corrected blinking component indicator or the corrected brain wave signal feature amount.
   (21)

A signal processing system including
a second information processing apparatus that performs an emotion estimation on a basis of a signal outputted from a first information processing apparatus,
the first information processing apparatus including a signal analysis unit that calculates a blinking component indicator and a brain wave signal feature amount on a basis of a blinking component and a brain wave component included in a brain wave signal actually measured by an electroencephalograph, and
the second information processing apparatus including
   a correction unit that performs a correction of the blinking component indicator or the brain wave signal feature amount in the actually measured brain wave signal on a basis of a correction table generated on a basis of a brain wave signal serving as a reference for the correction, the blinking component being superimposed on the brain wave signal feature amount, and
   an emotion estimation unit that performs the emotion estimation on a basis of at least the corrected blinking component indicator or the corrected brain wave signal feature amount.
      (22)

A signal processing system including:
a selection unit that selects a correction method of an emotion estimation on a basis of information regarding content, the correction method being in accordance with a context; and
an emotion estimation unit that performs the emotion estimation based on a blinking component indicator calculated on a basis of a blinking signal or a brain wave signal, in which
the emotion estimation unit performs the emotion estimation based on the blinking component indicator through the correction method selected by the selection unit.
   (23)

The signal processing system according to (22), in which
the information regarding the content includes at least one of metadata on the content or log data on the content, and
the context includes at least one of a genre of the content, a scene of the content, or elapsed time of the content.
   (24)

The signal processing system according to (22) or (23), in which
as the correction method of the emotion estimation, the selection unit selects a model parameter of an emotion estimation model in accordance with the context, or calculates a correction value for the blinking component indicator in accordance with the context, and
the emotion estimation unit performs the emotion estimation using the model parameter selected by the selection unit, or performs the emotion estimation based on the blinking component indicator corrected using the correction value for the blinking component indicator calculated by the selection unit.
   (25)

The signal processing system according to any one of (22) to (24), in which the selection unit selects, as the correction method of the emotion estimation, a correction method that reduces an error with respect to a predetermined reference relationship, in a case where the context becomes a context having a state where a relationship between blinking and an emotion is different from the predetermined reference relationship.

(26)

The signal processing system according to (25), in which the selection unit selects, as the correction method of the emotion estimation, the correction method that reduces the error with respect to the predetermined reference relationship, in a case where a scene that causes a perceptive activity with respect to a visual stimulus to be predominant is detected as the context.

(27)

The signal processing system according to (25) or (26), in which the selection unit selects, as the correction method of the emotion estimation, the correction method that reduces the error with respect to the predetermined reference relationship, in a case where a situation where a plurality of times of scene switching is included in an analysis window is detected as the context.

(28)

The signal processing system according to any one of (25) to (27), in which the selection unit selects, as the correction method of the emotion estimation, the correction method that reduces the error with respect to the predetermined reference relationship, in a case where a scene that arouses fear is detected as the context.

(29)

The signal processing system according to any one of (25) to (28), in which the selection unit selects, as the correction method of the emotion estimation, the correction method that reduces the error with respect to the predetermined reference relationship, in a case where a same scene as the context lasts for a long time period exceeding a threshold.

(30)

The signal processing system according to any one of (22) to (29), further including a mounting guide unit that estimates a mounted state of a device configured to measure the blinking signal or the brain wave signal, the mounting guide unit presenting information that encourages an improvement in the mounted state on a basis of the estimated mounted state.

The present application claims the benefit of Japanese Priority Patent Application JP 2022-183356 filed with the Japan Patent Office on Nov. 16, 2022, the entire contents of which are incorporated herein by reference.

It should be understood by those skilled in the art that various modifications, combinations, sub-combinations and alterations may occur depending on design requirements and other factors insofar as they are within the scope of the appended claims or the equivalents thereof.

1. A signal processing system comprising:
a signal analysis unit that calculates a blinking component indicator and a brain wave signal feature amount on a basis of a blinking component and a brain wave component included in a brain wave signal actually measured by an electroencephalograph;
a correction unit that performs a correction of the blinking component indicator or the brain wave signal feature amount in the actually measured brain wave signal on a basis of a correction table generated on a basis of a brain wave signal serving as a reference for the correction, the blinking component being superimposed on the brain wave signal feature amount; and
an emotion estimation unit that performs an emotion estimation on a basis of at least the corrected blinking component indicator or the corrected brain wave signal feature amount on which the blinking component is superimposed.

2. The signal processing system according to claim 1, wherein
the signal analysis unit calculates the blinking component indicator and the brain wave signal feature amount on which the blinking component is superimposed,
the correction unit corrects the brain wave signal feature amount on which the blinking component is superimposed, and
the emotion estimation unit performs the emotion estimation on a basis of the corrected brain wave signal feature amount on which the blinking component is superimposed.

3. The signal processing system according to claim 1, wherein
the signal analysis unit calculates the blinking component indicator and the brain wave signal feature amount on a basis of the blinking component and the brain wave component that are separated from the actually measured brain wave signal,
the correction unit performs the correction of the blinking component indicator, and
the emotion estimation unit performs the emotion estimation on a basis of the separated brain wave signal feature amount and the corrected blinking component indicator.

4. The signal processing system according to claim 1, wherein
the signal analysis unit calculates the blinking component indicator and the brain wave signal feature amount on which the blinking component is superimposed,
the correction unit
separates the blinking component from the brain wave signal feature amount on which the blinking component is superimposed, and
performs the correction of the blinking component indicator of the separated blinking component, and
the emotion estimation unit performs the emotion estimation on a basis of the separated brain wave signal feature amount and the corrected blinking component indicator.

5. The signal processing system according to claim 1, wherein
the signal analysis unit calculates the blinking component indicator and the brain wave signal feature amount on which the blinking component is superimposed,
the correction unit
separates a portion of the blinking component from the brain wave signal feature amount on which the blinking component is superimposed, and
performs the correction of the brain wave signal feature amount on which a portion of the blinking component is superimposed and the blinking component indicator of the separated blinking component, and
the emotion estimation unit performs the emotion estimation on a basis of the corrected blinking component indicator and the corrected brain wave signal feature amount on which the portion of the blinking component is superimposed.

6. The signal processing system according to claim 1, wherein the correction unit holds, out of the blinking component indicator serving as the reference for the correction and the brain wave signal feature amount on which the blinking component is superimposed and that serves as the reference for the correction, at least the blinking component indicator serving as the reference for the correction, the blinking component indicator and the brain wave signal feature amount being each calculated on a basis of a past actual measurement result of the brain wave signal serving as the reference for the correction or a simulation result of the brain wave signal serving as the reference for the correction.

7. The signal processing system according to claim 1, wherein the correction unit holds, as the correction table, a table indicating: a distribution of the blinking component indicator serving as the reference for the correction; or a distribution of the brain wave signal feature amount on which the blinking component is superimposed and that serves as the reference for the correction.

8. The signal processing system according to claim 1, wherein the correction unit
performs, using the correction table, a comparison between: the blinking component indicator serving as correction target data or the brain wave signal feature amount on which the blinking component is superimposed and that serves as the correction target data; and the blinking component indicator obtained on a basis of a past actual measurement result and serving as correction reference data or the brain wave signal feature amount on which the blinking component is superimposed, that is obtained on a basis of the past actual measurement result, and that serves as the correction reference data, and
calculates, on a basis of a result of the comparison, a correction coefficient that corrects a difference in a value of the blinking component indicator or the brain wave signal feature amount between pieces of data that are the correction target data and the correction reference data.

9. The signal processing system according to claim 8, wherein the emotion estimation unit performs the emotion estimation on a basis of at least: the blinking component indicator corrected on a basis of the correction coefficient; or the brain wave signal feature amount corrected on a basis of the correction coefficient.

10. The signal processing system according to claim 1, wherein the correction unit performs the correction of the blinking component indicator or the brain wave signal feature amount using, as additional information, biological information different from the brain wave signal outputted from a device mounted with the electroencephalograph, the blinking component being superimposed on the brain wave signal feature amount.

11. The signal processing system according to claim 1, further comprising a mounting guide unit that estimates a mounted state of a device mounted with the electroencephalograph, the mounting guide unit presenting information that encourages an improvement in the mounted state on a basis of the estimated mounted state.

12. The signal processing system according to claim 1, further comprising a selection unit that selects a correction method of the emotion estimation on a basis of information regarding content, the correction method being in accordance with a context, wherein
the emotion estimation unit performs the emotion estimation based on the blinking component indicator through the correction method selected by the selection unit.

13. The signal processing system according to claim 12, wherein
the information regarding the content comprises at least one of metadata on the content or log data on the content, and
the context comprises at least one of a genre of the content, a scene of the content, or elapsed time of the content.

14. The signal processing system according to claim 12, wherein
as the correction method of the emotion estimation, the selection unit selects a model parameter of an emotion estimation model in accordance with the context, or calculates a correction value for the blinking component indicator in accordance with the context, and
the emotion estimation unit performs the emotion estimation using the model parameter selected by the selection unit, or performs the emotion estimation based on the blinking component indicator corrected using the correction value for the blinking component indicator calculated by the selection unit.

15. The signal processing system according to claim 12, wherein the selection unit selects, as the correction method of the emotion estimation, a correction method that reduces an error with respect to a predetermined reference relationship, in a case where the context becomes a context having a state where a relationship between blinking and an emotion is different from the predetermined reference relationship.

16. The signal processing system according to claim 15, wherein the selection unit selects, as the correction method of the emotion estimation, the correction method that reduces the error with respect to the predetermined reference relationship, in a case where a scene that causes a perceptive activity with respect to a visual stimulus to be predominant is detected as the context.

17. The signal processing system according to claim 15, wherein the selection unit selects, as the correction method of the emotion estimation, the correction method that reduces the error with respect to the predetermined reference relationship, in a case where a situation where a plurality of times of scene switching is included in an analysis window is detected as the context.

18. The signal processing system according to claim 15, wherein the selection unit selects, as the correction method of the emotion estimation, the correction method that reduces the error with respect to the predetermined reference relationship, in a case where a scene that arouses fear is detected as the context.

19. The signal processing system according to claim 15, wherein the selection unit selects, as the correction method of the emotion estimation, the correction method that reduces the error with respect to the predetermined reference relationship, in a case where a same scene as the context lasts for a long time period exceeding a threshold.

20. A signal processing system comprising a second information processing apparatus that performs an emotion estimation on a basis of a signal outputted from a first information processing apparatus,
the first information processing apparatus including
a signal analysis unit that calculates a blinking component indicator and a brain wave signal feature amount on a basis of a blinking component and a brain wave component included in a brain wave signal actually measured by an electroencephalograph, and
a correction unit that performs a correction of the blinking component indicator or the brain wave signal feature amount in the actually measured brain wave signal on a basis of a correction table generated on a basis of a brain wave signal serving as a reference for the correction, the blinking component being superimposed on the brain wave signal feature amount, and
the second information processing apparatus including an emotion estimation unit that performs the emotion estimation on a basis of at least the corrected blinking component indicator or the corrected brain wave signal feature amount.

21. A signal processing system comprising
a second information processing apparatus that performs an emotion estimation on a basis of a signal outputted from a first information processing apparatus,
the first information processing apparatus including a signal analysis unit that calculates a blinking component indicator and a brain wave signal feature amount on a basis of a blinking component and a brain wave component included in a brain wave signal actually measured by an electroencephalograph, and
the second information processing apparatus including
a correction unit that performs a correction of the blinking component indicator or the brain wave signal feature amount in the actually measured brain wave signal on a basis of a correction table generated on a basis of a brain wave signal serving as a reference for the correction, the blinking component being superimposed on the brain wave signal feature amount, and
an emotion estimation unit that performs the emotion estimation on a basis of at least the corrected blinking component indicator or the corrected brain wave signal feature amount.

22. A signal processing system comprising:
a selection unit that selects a correction method of an emotion estimation on a basis of information regarding content, the correction method being in accordance with a context; and
an emotion estimation unit that performs the emotion estimation based on a blinking component indicator calculated on a basis of a blinking signal or a brain wave signal, wherein
the emotion estimation unit performs the emotion estimation based on the blinking component indicator through the correction method selected by the selection unit.

23. The signal processing system according to claim 22, wherein
the information regarding the content comprises at least one of metadata on the content or log data on the content, and
the context comprises at least one of a genre of the content, a scene of the content, or elapsed time of the content.

24. The signal processing system according to claim 22, wherein
as the correction method of the emotion estimation, the selection unit selects a model parameter of an emotion estimation model in accordance with the context, or calculates a correction value for the blinking component indicator in accordance with the context, and
the emotion estimation unit performs the emotion estimation using the model parameter selected by the selection unit, or performs the emotion estimation based on the blinking component indicator corrected using the correction value for the blinking component indicator calculated by the selection unit.

25. The signal processing system according to claim 22, wherein the selection unit selects, as the correction method of the emotion estimation, a correction method that reduces an error with respect to a predetermined reference relationship, in a case where the context becomes a context having a state where a relationship between blinking and an emotion is different from the predetermined reference relationship.

26. The signal processing system according to claim 25, wherein the selection unit selects, as the correction method of the emotion estimation, the correction method that reduces the error with respect to the predetermined reference relationship, in a case where a scene that causes a perceptive activity with respect to a visual stimulus to be predominant is detected as the context.

27. The signal processing system according to claim 25, wherein the selection unit selects, as the correction method of the emotion estimation, the correction method that reduces the error with respect to the predetermined reference relationship, in a case where a situation where a plurality of times of scene switching is included in an analysis window is detected as the context.

28. The signal processing system according to claim 25, wherein the selection unit selects, as the correction method of the emotion estimation, the correction method that reduces the error with respect to the predetermined reference relationship, in a case where a scene that arouses fear is detected as the context.

29. The signal processing system according to claim 25, wherein the selection unit selects, as the correction method of the emotion estimation, the correction method that reduces the error with respect to the predetermined reference relationship, in a case where a same scene as the context lasts for a long time period exceeding a threshold.

30. The signal processing system according to claim 22, further comprising a mounting guide unit that estimates a mounted state of a device configured to measure the blinking signal or the brain wave signal, the mounting guide unit presenting information that encourages an improvement in the mounted state on a basis of the estimated mounted state.
